(19) United States
(12) Patent Application Publication
(10) Pub. No.: US 2026/0340083 A1
(43) Pub. Date: Oct. 29, 2026

(54) DATA CLASSIFICATION AND MODEL TRAINING METHOD AND APPARATUS, DEVICE, AND STORAGE MEDIUM

(71) Applicant: TENCENT TECHNOLOGY (SHENZHEN) COMPANY LIMITED, Shenzhen (CN)

(72) Inventors: Yu ZHAO, Shenzhen (CN); Zitao XUAN, Shenzhen (CN); Chenchen QIN, Shenzhen (CN); Bing HE, Shenzhen (CN); Jianhua YAO, Shenzhen (CN)

(73) Assignee: TENCENT TECHNOLOGY (SHENZHEN) COMPANY LIMITED, Shenzhen (CN)

(21) Appl. No.: 19/692,283

(22) Filed: May 29, 2026

Related U.S. Application Data
(63) Continuation of application No. PCT/CN2025/098857, filed on Jun. 3, 2025.

(30) Foreign Application Priority Data
Aug. 26, 2024 (CN) 202411178661.3

(51) Int. Cl. G06F 18/2415; A61B 5/00

(52) U.S. Cl. CPC G06F 18/2415; A61B 5/7264; A61B 5/742

(57) ABSTRACT

A data classification method, apparatus, and computer-readable storage medium for early-stage cancer diagnosis using biological parameter analysis. The method acquires object data containing biological state parameter values for a classification object and candidate data from multiple reference objects. Feature information is extracted from both object and candidate data. Target candidate data satisfying correlation conditions with the object data is selected, and object feature information is adjusted using the selected candidate features. The adjusted features are classified to generate probability reference values indicating likelihood of belonging to preset cancer classifications. Results are displayed as diagnostic references for early-stage cancer detection. This approach enables personalized cancer risk assessment through comparative analysis of biological parameters against reference populations with feature correlation-based refinement.

### CROSS-REFERENCE TO RELATED APPLICATIONS

This application is a continuation application of International Application No. PCT/CN2025/098857 filed on Jun. 3, 2025 which claims priority to Chinese Patent Application No. 202411178661.3, filed with the China National Intellectual Property Administration on Aug. 26, 2024, the disclosures of each being incorporated by reference herein in their entireties.

### FIELD

The disclosure relates to the field of computer technologies, data classification and model training methods and apparatuses, a device, and a storage medium.

### BACKGROUND

With rapid development of artificial intelligence (AI) technologies and computer technologies, various data detection models have been widely used in some fields. In the field of medicine, accurate data detection can improve medical diagnosis. For example, detection of early-stage cancers can significantly increase a survival rate and reduce treatment costs, thus attracting widespread attention in academia and industry.

In the related art, such as detection of early-stage cancers, cancer detection may be performed based on cancer biological state parameters or based on an image analysis method. However, detection results of current detection methods are not accurate enough.

### SUMMARY

Provided are a data classification method and apparatus, a device, a storage medium, and a program product, which can implement accurate early-stage cancer diagnosis through biological parameter-based feature extraction, correlation-driven feature adjustment, and multi-reference object comparison.

According to some embodiments, a data classification method, performed by an electronic device, includes: acquiring object data of a classification object and Q pieces of candidate data of Q reference objects different from the classification object, wherein the object data includes parameter values of L biological state parameters of the classification object, each of the Q pieces of candidate data includes parameter values of the L biological state parameters of a corresponding reference object of the Q reference objects, and Q and L are each an integer greater than one; extracting object feature information from the object data and candidate feature information from the Q pieces of candidate data; selecting, based on the object feature information and the candidate feature information, at least one piece of target candidate data from among the Q pieces of candidate data, the at least one piece of target candidate data satisfying a preset correlation condition with the object data; adjusting the object feature information using the candidate feature information of the at least one piece of target candidate data to obtain adjusted object feature information of the object data; classifying the adjusted object feature information to obtain a classification result of the object data, the classification result including probability reference values indicating that the classification object belongs to a plurality of preset classifications; and displaying, at a terminal device, the classification result as a diagnostic reference for one or more early-stage cancers of the classification object.

According to some embodiments, a data classification apparatus, includes: at least one memory configured to store program code; and at least one processor configured to read the program code and operate as instructed by the program code, the program code including: acquisition code configured to cause at least one of the at least one processor to acquire object data of a classification object and Q pieces of candidate data of Q reference objects different from the classification object, wherein the object data includes parameter values of L biological state parameters of the classification object, each of the Q pieces of candidate data includes parameter values of the L biological state parameters of a corresponding reference object of the Q reference objects, and Q and Lare each an integer greater than one; extraction code configured to cause at least one of the at least one processor to extract object feature information from the object data and candidate feature information from the Q pieces of candidate data; selection code configured to cause at least one of the at least one processor to select, based on the object feature information and the candidate feature information, at least one piece of target candidate data from among the Q pieces of candidate data, the at least one piece of target candidate data satisfying a preset correlation condition with the object data; adjustment code configured to cause at least one of the at least one processor to adjust the object feature information using the candidate feature information of the at least one piece of target candidate data to obtain adjusted object feature information of the object data; classification code configured to cause at least one of the at least one processor to classify the adjusted object feature information to obtain a classification result of the object data, the classification result including probability reference values indicating that the classification object belongs to a plurality of preset classifications; and display code configured to cause at least one of the at least one processor to display, at a terminal device, the classification result as a diagnostic reference for one or more early-stage cancers of the classification object.

According to some embodiments, a non-transitory computer-readable storage medium, storing computer code which, when executed by at least one processor, causes the at least one processor to at least: acquire object data of a classification object and Q pieces of candidate data of Q reference objects different from the classification object, wherein the object data includes parameter values of L biological state parameters of the classification object, each of the Q pieces of candidate data includes parameter values of the L biological state parameters of a corresponding reference object of the Q reference objects, and Q and L are each an integer greater than one; extract object feature information from the object data and candidate feature information from the Q pieces of candidate data; select, based on the object feature information and the candidate feature information, at least one piece of target candidate data from among the Q pieces of candidate data, the at least one piece of target candidate data satisfying a preset correlation condition with the object data; adjust the object feature information using the candidate feature information of the at least one piece of target candidate data to obtain adjusted object feature information of the object data; classify the adjusted object feature information to obtain a classification result of the object data, the classification result including probability reference values indicating that the classification object belongs to a plurality of preset classifications; and display, at a terminal device, the classification result as a diagnostic reference for one or more early-stage cancers of the classification object.

### BRIEF DESCRIPTION OF THE DRAWINGS

To describe the technical solutions in the embodiments of the present disclosure more clearly, the following briefly describes the accompanying drawings required for describing the embodiments. Clearly, the accompanying drawings in the following descriptions show merely some embodiments of the present disclosure, and a person of ordinary skill in the art may still derive other drawings from these accompanying drawings without creative efforts.

FIG. 1 is a schematic diagram of an implementation environment of a data classification method according to some embodiments.

FIG. 2 is a schematic flowchart of a model training method according to some embodiments.

FIG. 3 is a schematic diagram of a data classification model.

FIG. 4 is a schematic diagram of a training sample and candidate samples.

FIG. 5 is a schematic diagram of a retrieval model.

FIG. 6A is a schematic diagram of selecting related candidate samples based on a similarity.

FIG. 6B is a schematic diagram of adjusting features of a training sample based on selected candidate samples.

FIG. 7 is a schematic diagram of performing data classification by using a classification module.

FIG. 8 is a schematic diagram of a retrieval module including R classification units.

FIG. 9 is a schematic flowchart of a model training method according to some embodiments.

FIG. 10 is a schematic diagram of a feature extraction unit.

FIG. 11 is a schematic diagram of classifying R pieces of adjusted feature information by using a classification module.

FIG. 12 is a schematic diagram of a network structure of a classification module.

FIG. 13 is a schematic diagram of performing data classification by using a MoE module.

FIG. 14 is a schematic flowchart of a data classification method according to some embodiments.

FIG. 15 is a schematic flowchart of a data classification method according to some embodiments.

FIG. 16 is a schematic flowchart of a data classification method according to some embodiments.

FIG. 17 is a schematic diagram of an overall process of data classification.

FIG. 18 is a schematic block diagram of a data classification apparatus according to some embodiments.

FIG. 19 is a schematic block diagram of a model training apparatus according to some embodiments.

FIG. 20 is a schematic block diagram of an electronic device according to some embodiments.

### DESCRIPTION OF EMBODIMENTS

To make the objectives, technical solutions, and advantages of the present disclosure clearer, the following further describes the present disclosure in detail with reference to the accompanying drawings. The described embodiments are not to be construed as a limitation to the present disclosure. All other embodiments obtained by a person of ordinary skill in the art without creative efforts shall fall within the protection scope of the present disclosure.

In the following descriptions, related “some embodiments” describe a subset of all possible embodiments. However, it may be understood that the “some embodiments” may be the same subset or different subsets of all the possible embodiments, and may be combined with each other without conflict. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. For example, the phrase “at least one of A, B, and C” includes within its scope “only A”, “only B”, “only C”, “A and B”, “B and C”, “A and C” and “all of A, B, and C.”

Technical solutions in embodiments of this application are clearly and completely described in the following with reference to the accompanying drawings in the embodiments of this application. Apparently, the described embodiments are merely some of rather than all of the embodiments of this application. Based on the embodiments in this application, all other embodiments obtained by a person of ordinary skilled in the art without creative efforts fall within the protection scope of this application.

In the specification, claims, and accompanying drawings of this application, the terms such as “first” and “second” are intended to distinguish similar objects, and are not necessarily intended to describe a order or sequence. Data used in such a way is interchangeable in a proper case, so that the embodiments of this application described herein can be implemented in an order other than those illustrated or described herein. In the embodiments of the present disclosure, “B corresponding to A” indicates that B is associated with A. In an implementation, B may be determined according to A. However, determining B according to A does not mean that B is determined according to only A, and B may be determined according to A and/or another information. In addition, the terms “include”, “have”, and any other variants mean to cover the non-exclusive inclusion. For example, a process, method, product, or device that includes a series of steps or units is not necessarily limited to the listed steps or units, but may include other steps or units not expressly listed or inherent to the process, method, product, or device. In the descriptions of this application, unless otherwise specified, “a plurality of” means two or more.

A data classification method provided in the embodiments of this application may be applied to various fields such as medical diagnosis and data detection, may implement classification of object data in multiple dimensions, and may improve classification accuracy. For example, classification reference results of a plurality of early-stage cancers may be obtained, thereby improving detection accuracy of the early-stage cancers.

For ease of understanding of the embodiments of this application, related concepts involved in the embodiments of this application are first described.

In medical detection, for example, detection of early-stage cancers can significantly increase a survival rate and reduce treatment costs, thus attracting widespread attention in academia and industry. Methods for detection of early-stage cancers mainly include: a detection method based on cancer biological state parameters (also known as biomarkers) and a detection method based on image analysis. The detection method based on cancer biological state parameters is to perform medical detection based on known cancer-related biological state parameters. biological state parameters available for cancer detection include prostate-antigen (PSA) for prostate cancer detection, CA-125 for ovarian cancer detection, and the like. The method based on image analysis is to perform auxiliary detection of related cancers by using various imaging technologies, such as low-dose computed tomography (CT) for lung cancer detection, colonoscopy for colorectal cancer detection, and Pap smear for cervical cancer detection.

However, the foregoing detection method based on cancer biological state parameters is merely limited to types of cancers. Moreover, biological state parameters related to the cancers are clearly known and lack sensitivity and specificity, and a cancer related to an unknown cancer biological state parameter cannot be detected. In addition, the method based on image analysis can target only cancers, cannot be applied to convenient and low-cost early cancer screening, and cannot perform pan-cancer analysis, and some cancers have no significant imaging features in early stages, resulting in a low detection rate when the method based on image analysis is used.

To resolve the foregoing technical problem, some embodiments provides a data classification method. When data is classified, object data of a classification object and Q pieces of candidate data of Q reference objects are first acquired. Object feature information of the object data and candidate feature information of the Q pieces of candidate data are respectively extracted. The object data includes parameter values of L biological state parameters of the classification object. The candidate data includes parameter values of L biological state parameters of a reference object. The classification object is different from the reference object. Then, at least one piece of target candidate data satisfying a preset correlation condition with the object data is selected from the Q pieces of candidate data based on the object feature information and the candidate feature information, and the object feature information is adjusted based on the candidate feature information of the at least one piece of target candidate data to obtain adjusted object feature information of the object data. Finally, the adjusted object feature information of the object data is classified to obtain a classification result of the object data. The classification result includes probability reference values indicating that the classification object belongs to a plurality of preset classifications. In some embodiments of this application, the at least one target candidate data selected from the Q pieces of candidate data is data of another reference object similar to the classification object. The object feature information of the object data is adjusted by using the candidate feature information of the at least one piece of target candidate data, so that features of similar objects can be integrated, making the adjusted object feature information of the object data richer and more comprehensive. Further, when the adjusted object feature information of the object data is used for data classification, a plurality of different classifications may be covered. For example, classification reference results of a plurality of different types of early-stage cancers can be obtained, and classification accuracy can be improved.

An implementation environment of the data classification method provided in some embodiments of this application is described below.

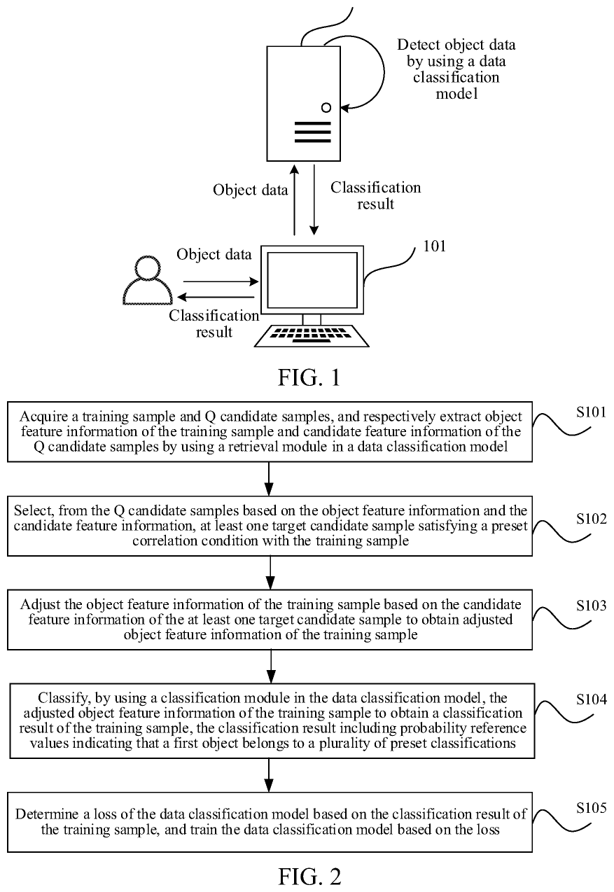

FIG. 1 is a schematic diagram of an implementation environment of a data classification method according to some embodiments. As shown in FIG. 1, the implementation environment includes: a terminal device 101 and a server 102.

The terminal device 101 is connected to the server 102 in a wired or wireless manner.

In some embodiments, a client of a data classification system is installed on the terminal device 101, and the server 102 may be understood as a server or a backend of the data classification system. A user may interact with the client of the data classification system installed on the terminal device 101. The server 102 stores a data classification model. The data classification model is configured for data classification, for example, for cancer classification in screening of early-stage cancers.

In some embodiments, the data classification model is first trained before data classification in some embodiments of this application. A process in which the server 102 trains the data classification model is described herein. The user may specify a training data set to the server 102 by using the terminal device. The server 102 acquires a training sample and Q candidate samples from the training data set. In some embodiments of this application, both the training sample and the candidate samples include parameter values of M biological state parameters (e.g., gene mutations) of different objects. In an example in which the biological state parameters are gene mutations, parameter values of the gene mutations include two values: Mutated and Not Mutated. For a certain gene, if the gene is mutated, a parameter value of the gene mutation is 1, and if the gene is not mutated, the parameter value of the gene mutation is 0. The server 102 respectively extracts object feature information of the training sample and candidate feature information of the Q candidate samples by using a retrieval module in a data classification model, the training sample including parameter values of L biological state parameters of a first object, the candidate samples including state values of L biological state parameters of a reference object different from the first object, and both Q and M being positive integers greater than 1; selects, from the Q candidate samples based on the object feature information and the candidate feature information, at least one target candidate sample satisfying a preset correlation condition with the training sample; adjusts the object feature information based on the candidate feature information of the at least one target candidate sample to obtain adjusted object feature information of the training sample; classifies, by using a classification module in the data classification model, the adjusted object feature information of the training sample to obtain a classification result of the training sample; and determines a loss of the data classification model based on a classification result of the training sample, and trains the data classification model based on the loss.

In some embodiments, the foregoing data classification model may be trained by using the terminal device 101, which is not limited in some embodiments of this application.

In some embodiments of this application, after the data classification model is trained, the data classification method in some embodiments of this application is performed by using the trained data classification model.

In some embodiments, the data classification method provided in some embodiments of this application is performed by the terminal device 101 and the server 102 together. For example, the user starts the client of the data classification system on the terminal device 101, the client displays an input interface of to-be-classified data, and the user enters to-be-processed object data into the input interface. The object data is parameter values of L biological state parameters of a classification object, for example, parameter values of L gene mutations of the classification object. The terminal device 101 transmits the to-be-classified object data to the server 102. The server 102 loads a candidate data set, and acquires Q pieces of candidate data from the candidate data set. Then, object feature information of the object data and candidate feature information of the Q pieces of candidate data are respectively extracted. For example, the server 102 respectively extracts the object feature information of the object data and the candidate feature information of the Q pieces of candidate data by using the retrieval module in the data classification model, the object data including parameter values of L biological state parameters of the classification object, and the candidate data including parameter values of L biological state parameters of a reference object different from the classification object, both Q and L being positive integers greater than 1; selects, from the Q pieces of candidate data based on the object feature information of the object data and the candidate feature information of the Q pieces of candidate data, at least one piece of target candidate data satisfying a preset correlation condition with the object data; adjusts the object feature information of the object data based on the candidate feature information of the at least one piece of target candidate data to obtain adjusted object feature information of the object data; and classifies the adjusted object feature information of the object data to obtain a classification result of the object data. For example, the adjusted object feature information of the object data is classified by using the classification module in the data classification model, to obtain the classification result of the object data. Then, the server 102 transmits the classification result of the object data (e.g., predicted values or probability reference values indicating that the classification object belongs to a plurality of preset classifications) to the terminal device 101 for display. In some embodiments of this application, the at least one target candidate data selected from the Q pieces of candidate data is data of another reference object similar to the first object, and the object feature information of the object data is adjusted by using the candidate feature information of the at least one piece of target candidate data, to integrate features of similar objects, making the adjusted object feature information of the object data richer and more comprehensive. Further, the adjusted object feature information of the object data is used for data classification, which can cover a plurality of different classifications and improve classification accuracy of the object data.

In some embodiments, the data classification method provided in some embodiments of this application is performed by the terminal device 101. In this case, the terminal device 101 may be any terminal device that can provide a data classification service. The server 102 may be any server that can provide a data downloading service. In an example, the foregoing server 102 stores a trained data classification model. A client of a data classification system is installed on the terminal device 101, and the client may request the data classification model from the server 102 based on a request of a user. For example, the terminal device 101 displays an input box of to-be-processed data, the user enters to-be-processed object data into the input interface, and the terminal device 101 transmits a request to the server 102 in response to an input operation of the user on the object data, to request the data classification model. The server 102 transmits the trained data classification model to the terminal device 101. The terminal device 101 loads a candidate data set, selects Q pieces of candidate data from the candidate data set, then respectively extracts object feature information of the object data and candidate feature information of the Q pieces of candidate data, for example, respectively extracts the object feature information of the object data and the candidate feature information of the Q pieces of candidate data by using the retrieval module in the data classification model, the object data including parameter values of L biological state parameters of a classification object, and the candidate data including parameter values of L biological state parameters of a reference object different from the classification object, both Q and L being positive integers greater than 1; selects, from the Q pieces of candidate data based on the object feature information and the candidate feature information, at least one piece of target candidate data satisfying a preset correlation condition with the object data; adjusts the object feature information of the object data based on the candidate feature information of the at least one piece of target candidate data to obtain adjusted object feature information of the object data; and classifies the adjusted object feature information of the object data to obtain a classification result of the object data. For example, the adjusted object feature information of the object data is classified by using the classification module in the data classification model, to obtain the classification result of the object data. Then, the terminal device 101 displays the classification result of the object data to the object. The classification result includes probability reference values indicating that the classification object belongs to a plurality of preset classifications.

In some embodiments, the foregoing terminal device 101 includes, but is not limited to, a desktop computer, a notebook computer, a smartphone, a tablet computer, an Internet of Things device, a portable wearable device, and the like. The Internet of Things device may be a smart speaker, a smart television, a smart air conditioner, a smart in-vehicle device, or the like. The portable wearable device may be a smart watch, a smart band, a head-mounted device, or the like. The terminal device is configured with a display apparatus. The display apparatus may be a display, a display screen, a touch screen, or the like. The touch screen may be a touchscreen, a touch panel, or the like.

In some embodiments, there may be one or more servers 102. When there are a plurality of servers, at least two servers are configured to provide different services and/or at least two servers are configured to provide the same service. For example, the same service is provided in a load balancing manner. This is not limited in some embodiments of this application. The foregoing server may be an independent physical server, or a server cluster or distributed system including a plurality of physical servers, or may be a cloud server providing basic cloud computing services such as a cloud service, a cloud database, cloud computing, a cloud function, cloud storage, a network service, cloud communication, a middleware service, a domain name service, a security service, a content delivery network (CDN), big data, and an AI platform. The server may be a blockchain node.

The implementation environment in some embodiments of this application includes, but is not limited to, that shown in FIG. 1.

The technical solution in some embodiments of this application is described in detail below by using some embodiments. The following several embodiments may be combined with each other, and the same or similar concept or process may not be described in detail again in some embodiments.

First, referring to FIG. 2, a training process of the data classification model in some embodiments of this application is described.

FIG. 2 is a schematic flowchart of a model training method according to some embodiments. Some embodiments of this application may be performed by an apparatus having a model training function, for example, a model training apparatus. The model training apparatus may be an electronic device such as the server 102 or the terminal device 101 shown in FIG. 1, or may be a system including the server 102 and 101 shown in FIG. 1. For ease of description, the method in some embodiments of this application is described in the following embodiment by using an electronic device as an example.

As shown in FIG. 2, the model training method in some embodiments of this application includes the following operations:

S101: Acquire a training sample and Q candidate samples, and respectively extract object feature information of the training sample and candidate feature information of the Q candidate samples by using a retrieval module in a data classification model.

The training sample includes parameter values of L biological state parameters of a first object, and the candidate samples include parameter values of L biological state parameters of a reference object different from the first object. For example, for an i^{th} candidate sample among the Q candidate samples, the i^{th} candidate sample may be understood as parameter values of L biological state parameters of an i^{th} reference object, the i^{th} object and the first object are different objects, both Q and L are positive integers greater than 1, and i takes values 1, 2, . . . , Q.

The data used in some embodiments of this application and the process of acquiring the data are in compliance with provisions of relevant laws and regulations.

The data classification model in some embodiments of this application may be applied to different fields, and is configured to classify data in different fields. Exemplarily, the method may be applied to the fields of biology and medicine to classify data related to the fields of biology and medicine. For example, the method is configured for auxiliary detection of early-stage cancers.

In the fields of biology and medicine, the first object and the reference object may represent different patients. The biological state parameters may include parameters such as a serological marker and a methylation state. In an example in which the biological state parameters are gene mutations, the training sample may include parameter values of L gene mutations of a patient 1, and the Q candidate samples may be parameter values of L gene mutations of Q patients. According to the method in some embodiments, probability reference valued indicating that the patient 1 belongs to a plurality of cancer classifications may be detected.

In some embodiments of this application, when the data classification model is used in different fields, the data classification model may be trained by using training samples in corresponding fields.

In some embodiments of this application, before training the data classification model, the electronic device first acquires a training data set. The training data set includes N training samples, which may be represented as {S₁, S₂, . . . , S_{N}}. Each training sample includes parameter values of L possible biological state parameters of an object. For example, an i^{th} training sample includes parameter values of L biological state parameters of an i^{th} object, and the i^{th} training sample may be represented as

$$\{ {M}_{1}^{i} , {M}_{2}^{i} , … , {M}_{L}^{i} \} , where {M}_{1}^{i}$$

denotes a parameter value of the first biological state parameter of the i^{th} object,

$${M}_{2}^{i}$$

denotes a parameter value of the second biological state parameter of the i^{th} object, and by analogy,

$${M}_{L}^{i}$$

denotes a parameter value of an L^{th} biological state parameter of the i^{th} object.

Exemplarily, the N training samples included in the training data set may be shown in Table 1:

TABLE 1

| | Biological | Biological | Biological | | Biological |
|---|---|---|---|---|---|
| | state | state | state | | state |
| | parameter 1 | parameter 2 | parameter 3 | . . . | parameter L |
| Object 1 | ${M}_{1}^{1}$ | ${M}_{2}^{1}$ | ${M}_{3}^{1}$ | . . . | ${M}_{L}^{1}$ |
| Object 2 | ${M}_{1}^{2}$ | ${M}_{2}^{2}$ | ${M}_{3}^{2}$ | . . . | ${M}_{L}^{2}$ |
| . . . | . . . | . . . | . . . | . . . | . . . |
| Object N | ${M}_{1}^{N}$ | ${M}_{2}^{N}$ | ${M}_{3}^{N}$ | . . . | ${M}_{L}^{N}$ |

Each row in Table 1 above represents one training sample. For example, the first row in Table 1 above is a training sample S₁, where the training sample S₁ includes parameter values of the L biological state parameters of the object 1

$$( {M}_{1}^{1} , {M}_{2}^{1} , {M}_{3}^{1} , … , {M}_{L}^{1} ) ,$$

and the second row in Table 1 above is a training sample S₂, where the training sample S₂ includes parameter values of the L biological state parameters of the object 2

$$( {M}_{1}^{2} , {M}_{2}^{2} , {M}_{3}^{2} , … , {M}_{L}^{2} ) .$$

Exemplarily, the training data set in some embodiments of this application may form a matrix with N rows and L columns, where each row of the matrix is one training sample.
types of the foregoing L biological state parameters are not limited in some embodiments of this application.

In a possible situation, the foregoing L biological state parameters may include gene mutations, where parameter values of the gene mutations may be understood as state values indicating whether a gene is mutated or not. In this case, the state values include 2 values: Mutated and Not Mutated. In some embodiments, 1 may be used to represent Mutated, and 0 may be used to represent Not Mutated. Certainly, 1 may be used to represent Not Mutated, and 0 may be used to represent Mutated. types and a quantity of the gene mutations are not limited in some embodiments of this application. Exemplarily, the gene mutations may include gene mutations such as ABL1, ACVR1, and AKT1. ABL1 is a proto-oncogene, ACVR1 is a gene encoding an activin type I receptor, and AKT1 is a thymoma viral proto-oncogene.

In a possible situation, the foregoing L biological state parameters may further include a serological marker, a methylation state, and the like. In this case, the parameter values of the biological state parameters may be determined according to a actual situation, which may be, for example, detection values of biological state parameters.

In some embodiments of this application, label information further may be determined for the foregoing N training samples. For each training sample, the label information of the training sample represents true values indicating that the training sample belongs to preset classifications. Assuming that the preset classifications include C classifications, in this case, the label information of the N training samples may be defined as {Y₁, Y₂, . . . , Y_{N}}, where Yᵢ∈{0, . . . , C}, and C represents a quantity of preset classifications. A value of C varies according to different training data sets.

In some embodiments, the foregoing L biological state parameters include L gene mutations, and the corresponding preset classifications include a plurality of cancer classifications.

In the data classification model in some embodiments of this application, an attempt is made to construct a mapping function Yᵢ=F(Sᵢ), and a plurality of preset classifications may be classified based on parameter values of L biological state parameters of any object. For example, when the foregoing preset classifications are a plurality of cancer categories, some embodiments of this application is used to develop a highly sensitive and multi-cancer early detection (MCED) model. The foregoing biological state parameters may relate to parameters such as a gene mutation, a serological marker, and a methylation state.

In some embodiments of this application, a process of training the data classification model by using each training sample in the foregoing training data set is basically consistent. For ease of description, one training sample is taken as an example herein to describe a model training process in some embodiments of this application.

In some embodiments of this application, for each training sample in the training data set, when the data classification model is trained by using the training sample, Q candidate samples corresponding to the training sample may be acquired.

A selection manner of acquiring the Q candidate samples corresponding to the training sample is not limited in some embodiments of this application.

In a possible implementation, for each of the N training samples, for example, an i^{th} training sample Sᵢ, the electronic selects, from N−1 training samples among the N training samples in the above training data set except the training sample Sᵢ, Q training samples as Q candidate samples corresponding to the training sample Sᵢ. For example, if Q is less than N−1, Q training samples may be randomly selected from the N−1 training samples as the Q candidate samples corresponding to the training sample Sᵢ. In another example, if Q is equal to N−1, the remaining N−1 training samples in the training data set except the training sample Sᵢ may be used as the Q candidate samples corresponding to the training sample Sᵢ.

In a possible implementation, when the data classification model is trained, multiple rounds of training are performed, and in each round of training, a batch of training samples is selected from the N training samples for the current round of training. In this case, in each round of training, the training sample Sᵢmay be understood as any training sample in a current batch of training samples. In this case, the Q candidate samples include all samples in the current batch of training samples except the training sample Sᵢ.

In a possible implementation, the electronic device may further load another data set. The another data set includes a plurality of candidate samples. Different candidate samples include parameter values of L biological state parameters of different objects. In this way, the electronic device may select, from the data set, the Q candidate samples corresponding to the training sample.

In some embodiments of this application, after acquiring the training sample and the Q candidate samples corresponding to the training sample, the electronic device trains the data classification model by using the training sample and the Q candidate samples.

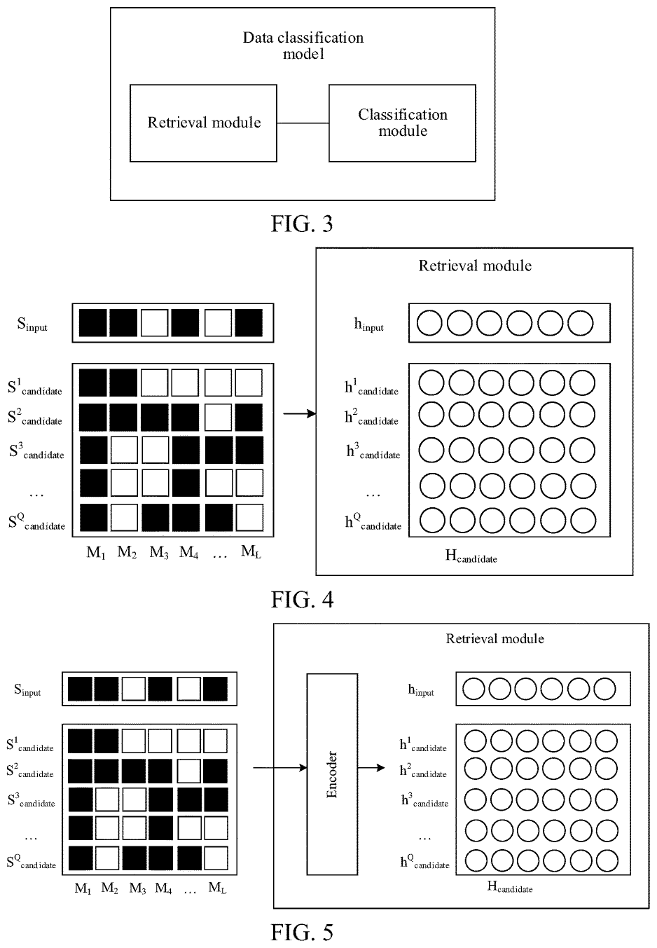

As shown in FIG. 3, the data classification model in some embodiments of this application includes a retrieval module and a classification module. A main function of the retrieval module is to select, from the Q candidate samples, at least one target candidate sample related to the training sample. In some embodiments, when it is determined whether a certain candidate sample is related to the training sample, a judgment may be made by determining whether the candidate sample and the training sample satisfy a preset correlation condition. Then, object feature information of the training sample is adjusted by using the at least one target candidate sample. Features of other relevant candidate samples are integrated by adjusting features of the training sample. In this way, accuracy of data classification can be improved when adjusted features of the training sample are used for subsequent data classification. The classification module is configured to classify the adjusted features of the training sample.

In some embodiments, for ease of description, a used training sample may be represented as Sᵢₙₚᵤₜ. Sᵢₙₚᵤₜ includes parameter values of L biological state parameters of an object, for example, the first object. Q candidate samples corresponding to the training sample Sᵢₙₚᵤₜ may be represented as S¹_{candidate}, S²_{candidate}, . . . , S^{Q}_{candidate}. The Q candidate samples include parameter values of L biological state parameters of other Q reference objects different from the first object. Exemplarily, FIG. 4 is a schematic diagram of the training sample Sᵢₙₚᵤₜ and 5 candidate samples when Lis equal to 6 and Q is equal to 5. Each small square in the training sample and the candidate samples in FIG. 4 represents an element value, that is, a parameter value of a certain biological state parameter of a certain object. Different colors of the small square represent different parameter values. In an example in which the biological state parameters are gene mutations, if the small square is dark (e.g., black), it indicates that the corresponding gene is mutated, and if the small square is light (e.g., white), it indicates that the gene is not mutated. Conversely, if the small square is dark, it indicates that the corresponding gene is not mutated, and if the small square is light, it indicates that the gene is mutated.

As shown in FIG. 4, the electronic device inputs the training sample and the Q candidate samples into the retrieval module, and the retrieval module respectively extracts object feature information of the training sample and candidate feature information of each candidate sample.

In some embodiments of this application, manners in which the retrieval module extracts the object feature information of the training sample and the candidate feature information of the Q candidate samples include at least the following several manners:

In Manner 1, the retrieval module does not consider the Q candidate samples when extracting the object feature information of the training sample, and does not consider the training sample and other candidate samples among the Q candidate samples when extracting the candidate feature information of each of the Q candidate samples. The manner 1 may be understood as that the electronic device inputs the training sample and the Q candidate samples, a total of Q+1 samples, one by one into the retrieval module for feature extraction, and during the feature extraction, no feature interaction or fusion occurs between these samples.

In Manner 2, the retrieval module considers the Q candidate samples when extracting the object feature information of the training sample, and does not consider the training sample and other candidate samples among the Q candidate samples when extracting the candidate feature information of each of the Q candidate samples. The manner 2 may be understood as that the electronic device inputs the training sample and the Q candidate samples, a total of Q+1 samples, together into the retrieval module for feature extraction, and during the feature extraction, feature interaction or fusion occurs between these samples.

A network structure of the retrieval module is not limited in some embodiments of this application.

In an example, as shown in FIG. 5, the retrieval module includes an encoder, and the encoder is configured for feature extraction. In this way, the electronic device may input the training sample and the Q candidate samples into the encoder for feature extraction, to obtain the object feature information of the training sample and the candidate feature information of each of the Q candidate samples.

Exemplarily, a process in which the electronic device extracts the object feature information of the training sample and the candidate feature information of the Q candidate samples by using the encoder may be expressed in the following Formula (1):

hᵢₙₚᵤₜ=E(Sᵢₙₚᵤₜ)hⁱ_{candidate}=E(Sⁱ_{candidate}),i∈Q_{candidate} (1)

where E represents the encoder, and hᵢₙₚᵤₜ∈R^{1×d} represents the object feature information of the training sample Sᵢₙₚᵤₜ, hⁱ_{candidate}∈R^{1×d} represents candidate feature information of an i^{th} candidate sample Sⁱ_{candidate}, and Q_{candidate} represents a candidate sample set including Q candidate samples. d represents a hidden dimension, that is, the object feature information hᵢₙₚᵤₜ of the training sample is a d-dimensional vector. Similarly, the candidate feature information hⁱ_{candidate} of the i^{th} candidate sample is also a d-dimensional vector.

In some embodiments of this application, based on the above operations, the electronic device acquires a training sample and Q candidate samples corresponding to the training sample, and extracts object feature information of the training sample and candidate feature information of each of the Q candidate samples by using the retrieval module in the data classification model. Then, operation S102 below is performed.

S102: Select, from the Q candidate samples based on the object feature information and the candidate feature information, at least one target candidate sample satisfying a preset correlation condition with the training sample.

In some embodiments of this application, to improve accuracy of data classification, at least one target candidate sample satisfying a preset correlation condition with the training sample is selected from the Q candidate samples. The target candidate sample is also referred to as a sample related to the training sample. Further, features of the training sample are adjusted by using features of the at least one target candidate sample. In this way, adjusted features of the training sample not only include features of the first object, but also include features of another object similar to the first object, so that the features of the first object are richer and more comprehensive. In this way, when data classification is performed based on rich and comprehensive feature information of the first object, accuracy of data classification can be improved.

A process in which the electronic device selects the at least one target candidate sample related to the training sample from the Q candidate samples is described below.

In some embodiments of this application, the electronic device selects, from the Q candidate samples based on the object feature information of the training sample and the candidate feature information of each of the Q candidate samples, the at least one target candidate sample related to the training sample.

The preset correlation condition is configured for determining whether a candidate sample and a training sample are correlated with each other. The preset correlation condition may be whether clustering results of the candidate sample and the training sample belong to the same cluster, or whether a similarity between the candidate sample and the training sample satisfy a set condition (e.g., greater than a preset similarity threshold, or selecting top N candidate samples with the highest similarity from the Q candidate samples). Essentially, a clustering algorithm is to classify samples by measuring similarities in a feature space, and samples belonging to the same cluster have a higher similarity. The similarity is configured for representing a correlation between the candidate sample and the training sample. A higher similarity indicates a stronger correlation therebetween.

In some embodiments, the electronic device clusters the object feature information of the training sample and the candidate feature information of the Q candidate samples, and determines a candidate sample that is among the Q candidate samples and belongs to the same cluster as the training sample as the target candidate sample satisfying the preset correlation condition with the training sample, that is, the target candidate sample related to the training sample. A similarity between the training sample and the candidate sample that belong to the same cluster is higher. Therefore, the candidate sample that is among the Q candidate samples and belongs to the same cluster as the training sample is determined as the target candidate sample related to the training sample.

In some embodiments, S102 includes the following operations S102-A and S102-B:

S102-A: Determine a similarity between the training sample and each of the Q candidate samples based on the object feature information of the training sample and the candidate feature information of the Q candidate samples.

S102-B: Select the at least one target candidate sample from the Q candidate samples based on the similarities between the training sample and the Q candidate samples.

In this implementation, for each of the Q candidate samples, a similarity between the candidate sample and the training sample is determined based on the candidate feature information of the candidate sample and the object feature information of the training sample.

A manner in which the electronic device determines the similarity between the candidate sample and the training sample based on the candidate feature information of the candidate sample and the object feature information of the training sample is not limited in some embodiments of this application.

In a possible implementation, the electronic device calculates a distance between the object feature information of the candidate sample and the candidate feature information of the training sample by using a distance calculation manner such as a cosine distance or an L2 distance, and then determines the similarity between the candidate sample and the training sample based on the distance. A larger distance indicates that the training sample and the candidate sample are less similar and a corresponding similarity value is smaller. A smaller distance indicates that the training sample and the candidate sample are more similar and the corresponding similarity value is larger. In an example, a reciprocal of the distance between the candidate feature information of the candidate sample and the object feature information of the training sample may be determined as the similarity between the candidate sample and the training sample.

In a possible implementation, S102-A may include the following operations S102-A1 to S102-A3:
S102-A1: Project, for a q^{th} candidate sample among the Q candidate samples, candidate feature information of the q^{th} candidate sample by using a first projection matrix, to obtain projection feature information of the q^{th} candidate sample, q taking values 1, 2, . . . , Q.
S102-A2: Project the object feature information of the training sample by using the first projection matrix, to obtain projection feature information of the training sample.
S102-A3: Determine a distance between the projection feature information of the training sample and the projection feature information of the q^{th} candidate sample as a similarity between the training sample and the q^{th} candidate sample.

In this implementation, when the similarity between the candidate sample and the training sample is calculated, the candidate feature information of the candidate sample and the object feature information of the training sample are projected by using a projection matrix, to obtain projection feature information of the candidate sample and the training sample, and then the similarity between the candidate sample and the training sample is calculated by using the projection feature information.

Specifically, for the q^{th} candidate sample among the Q candidate samples, the candidate feature information of the q^{th} candidate sample is projected by using the first projection matrix Wₖ. For ease of description, the projected candidate feature information is denoted as the projection feature information of the q^{th} candidate sample. Similarly, the object feature information of the training sample is projected by using the first projection matrix Wₖ, to obtain projection feature information of the training sample. Then, the distance between the projection feature information of the training sample and the projection feature information of the q^{th} candidate sample is calculated, and then the distance is determined as the similarity between the training sample and the q^{th} candidate sample.

In an example, the electronic device uses the L2 distance as the similarity between the projection feature information of the training sample and the projection feature information of the q^{th} candidate sample. The L2 distance is also referred to as a Euclidean distance. In this case, a calculation formula of the similarity is shown in Formula (2):

$$Sim ( {h}_{input} , {h}_{candidate}^{q} ) = {L}_{2} ( {W}_{k} ( {h}_{input} ) , {W}_{k} ( {h}_{candidate}^{q} ) ) \qquad ( 2 )$$

where

$$Sim ( {h}_{input} , {h}_{candidate}^{q} )$$

represents the similarity between the projection feature information of the training sample and the projection feature information of the q^{th} candidate sample, Wₖ∈R^{d×d} denotes the first projection matrix,

$${h}_{candidate}^{q}$$

denotes the candidate feature information of the q^{th} candidate sample, Wₖ(hᵢₙₚᵤₜ) denotes the projection feature information of the training sample, and

$${W}_{k} ( {h}_{candidate}^{q} )$$

denotes the projection feature information of the q^{th} candidate sample.

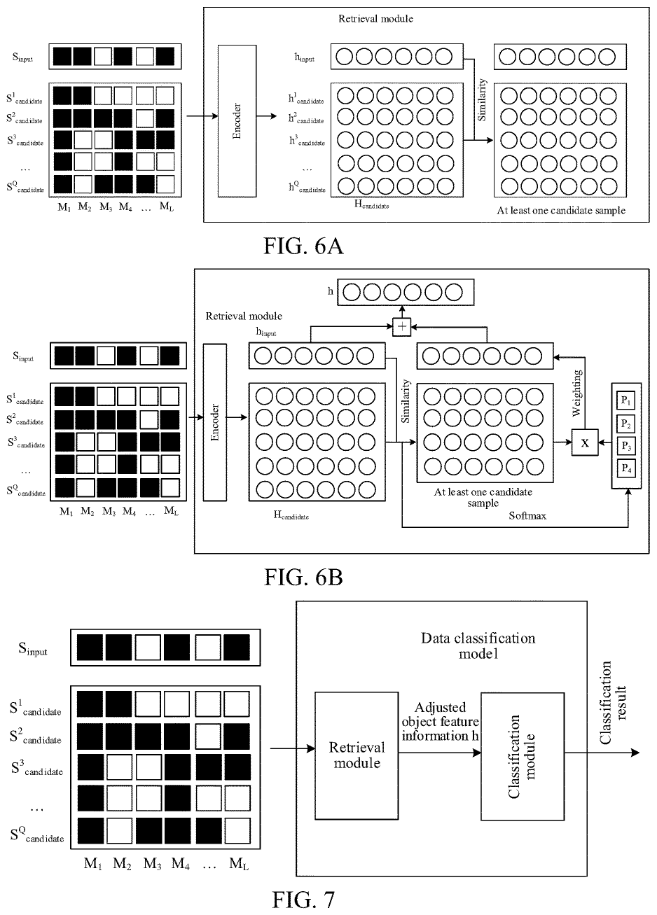

As shown in FIG. 6A, the electronic device may determine a similarity between each of the Q candidate samples and the training sample based on the foregoing method. In some embodiments of this application, the electronic device selects, from the Q candidate samples based on the similarity between each of the Q candidate samples and the training sample, the at least one target candidate sample related to the training sample. In an example, the electronic device selects, from the Q candidate samples, at least one candidate sample with a maximum similarity to the training sample (e.g., top k candidate samples with the greatest similarity as shown in FIG. 6) as the at least one target candidate sample related to the training sample.

S103: Adjust the object feature information of the training sample based on the candidate feature information of the at least one target candidate sample to obtain adjusted object feature information of the training sample.

In some embodiments of this application, adjusting the object feature information of the training sample may include expanding (or supplementing) the object feature information of the training sample to enrich the object feature information of the training sample, so that the adjusted object feature information includes not only feature information of the first object but also feature information of another object related to the first object. Adjusting the object feature information of the training sample may further include modifying or deleting an object feature of the training sample, for example, deleting or modifying some incorrect feature information. The adjusted object feature information is more accurate by deleting or modifying the incorrect feature information.

A manner in which the electronic device adjusts the object feature information of the training sample based on the candidate feature information of the at least one target candidate sample to obtain the adjusted object feature information of the training sample is not limited in some embodiments of this application.

In some embodiments, the electronic device may fuse the object feature information of the training sample with the candidate feature information of the at least one target candidate sample to obtain the adjusted object feature information of the training sample. For example, a weight of the at least one target candidate sample is determined based on a similarity between the at least one target candidate sample and the training sample, and then weighted processing is performed on the object feature information of the training sample and the candidate feature information of the at least one target candidate sample based on the weight to obtain the adjusted object feature information of the training sample.

In some embodiments, the electronic device may compare and analyze the object feature information of the training sample and the candidate feature information of the at least one target candidate sample, to determine whether the object feature information of the training sample may be modified or deleted, and if modification or deletion is needed, delete or modify the object feature information of the training sample according to the candidate feature information of the at least one target candidate sample to obtain the adjusted object feature information of the training sample. By deleting or modifying incorrect feature information, the adjusted object feature information is more accurate.

In some embodiments, S103 includes the following operations S103-A and S103-B:
S103-A: Determine, for a kth target candidate sample among the at least one target candidate sample, a distance between projection feature information of the training sample and projection feature information of the kth target candidate sample, and determine, based on the distance, first adjustment information of the kth target candidate sample for the training sample, k taking values 1, 2, . . . , K, and K being a quantity of target candidate samples satisfying the preset correlation condition with the training sample, that is, a quantity of target candidate samples related to the training sample.
S103-B: Adjust the object feature information of the training sample based on first adjustment information of the at least one target candidate sample for the training sample to obtain the adjusted object feature information of the training sample.

In this implementation, adjustment information of a candidate sample for the training sample is determined in a hidden layer of the data classification model by using a distance between the training sample and the at least one target candidate sample. Specifically, for the k^{th} target candidate sample among the at least one target candidate sample, the distance between the projection feature information of the training sample and the projection feature information of the k^{th} target candidate sample is determined, and the first adjustment information of the k^{th} target candidate sample for the training sample is determined based on the distance.

A manner of determining the first adjustment information of the k^{th} target candidate sample for the training sample is not limited in some embodiments of this application.

In some embodiments, the electronic device may determine the distance between the projection feature information of the training sample and the projection feature information of the k^{th} target candidate sample as the first adjustment information of the k^{th} target candidate sample for the training sample.

In some embodiments, the electronic device performs activation processing on the distance between the projection feature information of the training sample and the projection feature information of the k^{th} target candidate sample, then performs dropout processing, and performs lossless linear transformation on a result of the dropout processing, to obtain second adjustment information of the k^{th} target candidate sample for the training sample. Then, the second adjustment information of the k^{th} target candidate sample for the training sample is determined as the first adjustment information of the k^{th} target candidate sample for the training sample.

Exemplarily, the electronic device determines the second adjustment information by using the following Formula (3):

$${V}_{2} ( {h}_{input} , {h}_{candidate}^{k} ) = {W}_{T} ( {L}_{1} ( ( {h}_{input} ) , {W}_{k} ( {h}_{candidate}^{k} ) ) ) \qquad ( 3 )$$

where

$${V}_{2} ( {h}_{input} , {h}_{candidate}^{k} ) ∈ {R}^{1 \times d}$$

denotes the second adjustment information of the k^{th} target candidate sample for the training sample,

$${h}_{candidate}^{k}$$

denotes the k^{th} target candidate sample,

$${L}_{1} ( ( {h}_{input} ) , {W}_{k} ( {h}_{candidate}^{k} ) ) ∈ ( 1 \times d )$$

denotes an L1 distance between the projection feature information of the training sample and the projection feature information of the k^{th} target candidate sample, W_{T}(·)=LinearWithoutBias(Dropout(ReLU(Linear(·)))), Linear(·) denotes linear processing, ReLU denotes an activation function, Dropout (·) denotes dropout processing, and LinearWithoutBias(·) denotes lossless linear transformation. The L1 distance is also referred to as a Manhattan distance.

The foregoing manner of processing the distance between the projection feature information of the training sample and the projection feature information of the k^{th} target candidate sample to obtain the second adjustment information of the k^{th} target candidate sample for the training sample is merely an example. The electronic device may process the distance between the projection feature information of the training sample and the projection feature information of the k^{th} target candidate sample to obtain the second adjustment information of the k^{th} target candidate sample for the training sample in another manner.

In some embodiments, since the at least one target candidate sample is similar to the training sample, label information of the at least one target candidate sample may further provide additional information for the features of the training sample. Based on this, in S103-A, the determining, based on the distance, first adjustment information of the k^{th} target candidate sample for the training sample includes the following operations S103-A1 and S103-A2:

S103-A1: Perform linear activation processing on the distance between the projection feature information of the training sample and the projection feature information of the k^{th} target candidate sample, then perform dropout processing, and perform lossless linear transformation on a result of the dropout processing, to obtain second adjustment information of the k^{th} target candidate sample for the training sample.

S103-A2: Determine first adjustment information of the k^{th} target candidate sample for the training sample based on the second adjustment information of the k^{th} target candidate sample for the training sample and label information of the k^{th} target candidate sample.

For a process of performing linear activation processing on the distance between the projection feature information of the training sample and the projection feature information of the k^{th} target candidate sample, then performing dropout processing, and performing lossless linear transformation on the result of the dropout processing, to obtain the second adjustment information of the k^{th} target candidate sample for the training sample, refer to the description in the foregoing embodiments. Details are not described herein again.

Then, the electronic device determines the first adjustment information of the k^{th} target candidate sample for the training sample based on the second adjustment information of the k^{th} target candidate sample for the training sample and the label information of the k^{th} target candidate sample.

In a possible implementation, the electronic device determines a sum of the second adjustment information of the k^{th} target candidate sample for the training sample and the label information of the k^{th} target candidate sample as the first adjustment information of the k^{th} target candidate sample for the training sample.

In a possible implementation, the label information of the k^{th} target candidate sample is projected by using a second projection matrix, to obtain label projection information of the k^{th} target candidate sample; and a sum of the second adjustment information of the k^{th} target candidate sample for the training sample and the label projection information of the k^{th} target candidate sample is determined as the first adjustment information of the k^{th} target candidate sample for the training sample.

Exemplarily, the electronic device determines, by using the following Formula (4), the first adjustment information of the k^{th} target candidate sample for the training sample:

$$V ( {h}_{input} , {h}_{candidate}^{k} , {Y}_{k} ) = {W}_{T} ( {L}_{1} ( ( {h}_{input} ) , {W}_{k} ( {h}_{candidate}^{k} ) ) ) + {W}_{v} ( {Y}_{k} ) \qquad ( 4 )$$

where

$$V ( {h}_{input} , {h}_{candidate}^{k} , {Y}_{k} ) ∈ {R}^{1 \times d}$$

denotes the first adjustment information of the k^{th} target candidate sample for the training sample, Wᵥ∈R^{d×d} denotes the second projection matrix, Yₖ denotes the label information of the k^{th} target candidate sample, and Wᵥ(Yₖ) represents the label projection information of the k^{th} target candidate sample.

A process in which the electronic device determines the first adjustment information of the k^{th} target candidate sample among the at least one target candidate sample for the training sample is described above. With reference to the above operations, the electronic device may determine first adjustment information of each of the at least one target candidate sample for the training sample, and then perform the above operation S103-B to adjust the object feature information of the training sample based on the first adjustment information of the at least one target candidate sample for the training sample to obtain the adjusted object feature information of the training sample.

A manner of adjusting the object feature information of the training sample based on the first adjustment information of the at least one target candidate sample for the training sample to obtain the adjusted object feature information of the training sample is not limited in some embodiments of this application.

In some embodiments, the electronic device adds a sum or an average value of the first adjustment information of the at least one target candidate sample for the training sample to the object feature information of the training sample to obtain the adjusted object feature information of the training sample.

In some embodiments, S103-B includes the following operations S103-B1 to S103-B3:
S103-B1: Determine, for the k^{th} target candidate sample among the at least one target candidate sample, a weight of the k^{th} target candidate sample based on a similarity between the training sample and the k^{th} target candidate sample.
S103-B2: Perform, based on a weight of the at least one target candidate sample, weighted processing on the first adjustment information of the at least one target candidate sample for the training sample to obtain weighted adjustment information.
S103-B3: Determine the adjusted object feature information of the training sample based on the object feature information of the training sample and the weighted adjustment information.

In this implementation, as shown in FIG. 6B, for each of the at least one target candidate sample, for example, the k^{th} target candidate sample, the weight of the k^{th} target candidate sample is determined based on the similarity between the k^{th} target candidate sample and the training sample. The weight is positively correlated with the similarity. That is, a higher similarity indicates a larger weight, and conversely, a lower similarity indicates a smaller weight.

A manner in which the electronic device determines the weight of the k^{th} target candidate sample based on the similarity sim between the k^{th} target candidate sample and the training sample is not limited in some embodiments of this application. For example, the similarity sim between the k^{th} target candidate sample and the training sample is determined as the weight of the k^{th} target candidate sample.

In a possible implementation, as shown in FIG. 6, normalization processing is performed on the similarity sim between the k^{th} target candidate sample and the training sample to obtain the weight of the k^{th} target candidate sample.

For example, the electronic device determines the weight of the k^{th} target candidate sample by using the following Formula (5):

Pₖ=Softmax(Sim(hᵢₙₚᵤₜ,h^{k}_{candidate})) (5)

where Pₖ denotes the weight of the k^{th} target candidate sample, Sim(hᵢₙₚᵤₜ, h^{k}_{candidate}) denotes the similarity between the training sample and the k^{th} target candidate sample, and Softmax denotes a normalization processing function.

Based on the above operations, the electronic device may determine a weight of each of the at least one target candidate sample. As shown in FIG. 6B, based on the weight of the at least one target candidate sample, weighted processing is performed on the first adjustment information of the at least one target candidate sample for the training sample to obtain weighted adjustment information. Then, the adjusted object feature information of the training sample is determined based on the object feature information of the training sample and the weighted adjustment information. For example, as shown in FIG. 6, a sum of the object feature information of the training sample and the weighted adjustment information is determined as the adjusted object feature information of the training sample.

Exemplarily, the electronic device may determine the adjusted object feature information of the training sample by using the following Formula (6):

$$h = {h}_{input} + {\sum }_{k = 0}^{K} {P}_{k} \cdot V ( {h}_{input} , {h}_{candidate}^{k} , {Y}_{k} ) \qquad ( 6 )$$

where h denotes the adjusted object feature information of the training sample, hᵢₙₚᵤₜ denotes the object feature information of the training sample (that is, the object feature information of the training sample before adjustment), Pₖ denotes the weight of the k^{th} target candidate sample among the at least one target candidate sample (which may also be referred to as a normalized similarity),

$${h}_{candidate}^{k}$$

denotes the candidate feature information of the k^{th} target candidate sample, Yₖ denotes the label information of the k^{th} target candidate sample,

$$V ( {h}_{input} , {h}_{candidate}^{k} , {Y}_{k} )$$

denotes adjustment information of the k^{th} target candidates sample for the training sample, and

$${\sum }_{k = 0}^{K} {P}_{k} \cdot V ( {h}_{input} , {h}_{candidate}^{k} , {Y}_{k} )$$

denotes the weighted adjustment information.

S104: Classify, by using a classification module in the data classification model, the adjusted object feature information of the training sample to obtain a classification result of the training sample, the classification result including probability reference values indicating that a first object belongs to a plurality of preset classifications.

In some embodiments of this application, after determining the adjusted object feature information of the training sample based on the above operations, as shown in FIG. 7, the electronic device inputs the adjusted object feature information of the training sample into the classification module for data classification, to obtain a classification result of the training sample. The classification result includes probability reference values indicating that the first object belongs to a plurality of preset classifications.

When applied to different data classification scenarios, the plurality of preset classifications are different. For example, if the L biological state parameters include L gene mutations, the classification result includes probability reference values indicating that the first object belongs to a plurality of cancer classifications. The probability reference values indicating that the first object belongs to the plurality of preset classifications are configured for representing degrees of probability that the first object belongs to the preset classifications. Exemplarily, a sum of the probability reference values indicating that the first object belongs to the plurality of preset classifications is equal to 1.

S105: Determine a loss of the data classification model based on the classification result of the training sample, and train the data classification model based on the loss.

As can be known from above, in some embodiments of this application, the label information of the training sample has been acquired when the data classification model is trained by using the training sample. In this way, the electronic device inputs the training sample into the data classification model for data classification, to obtain a classification result of the training sample outputted by the data classification model, and then determines the loss of the data classification model based on a deviation between the classification result of the training sample and the label information of the training sample. A type of a loss function used when the electronic device determines the loss of the data classification model based on the classification result of the training sample and the label information of the training sample is not limited in some embodiments of this application. The classification result includes probability reference values indicating that the first object belongs to a plurality of cancer classifications, a label of the training sample is an actual probability that the first object belongs to the plurality of preset classifications, and the loss of the data classification model is determined based on the probability reference values indicating that the first object belongs to the plurality of cancer classifications and the actual probability. After the loss is determined, parameters in the data classification model are updated based on the loss. Then, one training sample is reselected from the training data set, and the data classification model with updated parameters is iteratively trained. In this way, training is repeated multiple times until a model training end condition is reached. The model training end condition may include a quantity of times of training reaching a preset quantity of times and/or a loss of the model reaching a preset value.

In the model training method provided in some embodiments of this application, a data classification model is trained, and the data classification model includes a retrieval module and a classification module. During model training, a training sample and Q candidate samples are first acquired. Object feature information of the training sample and candidate feature information of the Q candidate samples are respectively extracted by using the retrieval module in the data classification model. The training sample includes parameter values of L biological state parameters of a first object. The candidate samples include parameter values of L biological state parameters of a reference object different from the first object. Then, at least one target candidate sample satisfying a preset correlation condition with the training sample is selected from the Q candidate samples based on the object feature information of the training sample and the candidate feature information of the Q candidate samples. Then, the object feature information of the training sample is adjusted based on the candidate feature information of the at least one target candidate sample to obtain adjusted object feature information of the training sample. Finally, the adjusted object feature information of the training sample is classified by using the classification module in the data classification model, to obtain a classification result of the training sample. Then, a loss of the data classification model is determined based on a classification result of the training sample, and the data classification model is trained based on the loss. In some embodiments of this application, the at least one target candidate sample selected from the Q candidate samples is a sample of another object similar to the first object. The object feature information of the training sample is adjusted by using the candidate feature information of the at least one target candidate sample, so that features of similar objects can be integrated, making the adjusted object feature information of the training sample richer and more comprehensive. When the adjusted object feature information of the training sample is used for data classification, a plurality of different classifications may be covered. For example, classification reference results of a plurality of different types of early-stage cancers can be obtained, and classification accuracy can be improved.

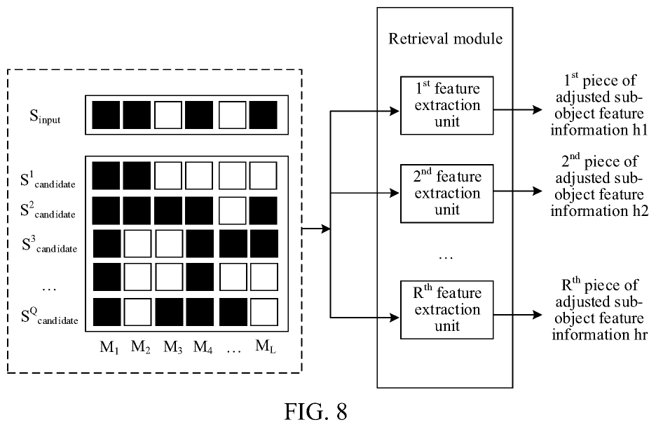

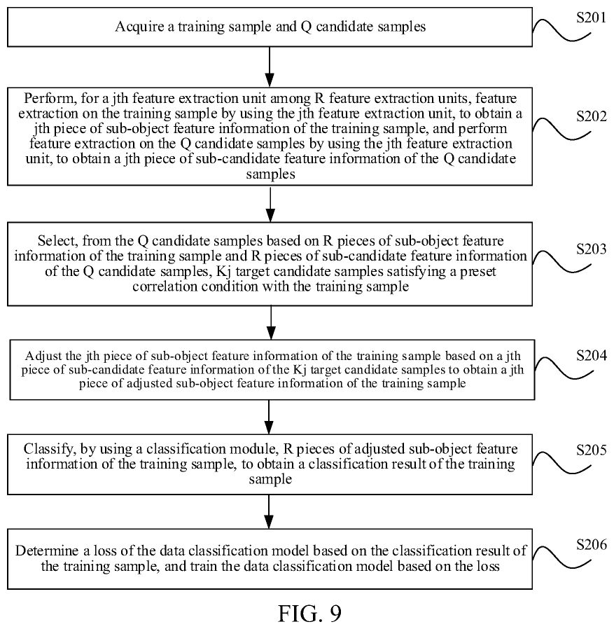

An overall training process of the data classification model is described hereinabove. In some embodiments, to further improve the accuracy of data classification, the training sample may be retrieved multiple times by using the retrieval module, to project the training sample to d dimensions by using different linear projections, so as to further extract features of the training sample and improve richness of the extracted features. In this case, as shown in FIG. 8, the retrieval module in some embodiments of this application includes R feature extraction units. The model training method in some embodiments of this application is further described below with reference to FIG. 9.

FIG. 9 is a schematic flowchart of a model training method according to some embodiments.

As shown in FIG. 9, a training process of a data detection model in some embodiments of this application includes the following operations:

S201: Acquire a training sample and Q candidate samples.

The training sample is parameter values of L biological state parameters of a first object, and the candidate samples are parameter values of L biological state parameters of a reference object different from the first object.

For a implementation process of S201 above, refer to the related description of S101 above. Details are not described herein again.

S202: Perform, for a j^{th} feature extraction unit among R feature extraction units, feature extraction on the training sample by using the j^{th} feature extraction unit, to obtain a j^{th} piece of sub-object feature information of the training sample, and perform feature extraction on the Q candidate samples by using the j^{th} feature extraction unit, to obtain a j^{th} piece of sub-candidate feature information of the Q candidate samples.

As shown in FIG. 8, the retrieval module in some embodiments of this application includes R feature extraction units. The R feature extraction units run in parallel. By separately processing the inputted training sample and Q candidate samples by using the R feature extraction units, R pieces of sub-object feature information of the training sample and R pieces of sub-object feature information of each candidate sample may be obtained. That is, the object feature information of the training sample includes R pieces of sub-object feature information, and the candidate feature information of each candidate sample includes R pieces of sub-candidate feature information.

In a possible implementation, the R pieces of sub-object feature information extracted by the R feature extraction units are concatenated to obtain the object feature information of the training sample, and the R pieces of sub-candidate feature information extracted by the R feature extraction units are concatenated to obtain the candidate feature information of the candidate sample. Exemplarily, concatenation may be performed by using a concat function.

In some embodiments of this application, processes of processing the training sample and the Q candidate samples corresponding to the training sample by each of the R feature extraction units are consistent. For ease of description, the j^{th} feature extraction unit is taken as an example herein for description.

In some embodiments of this application, a data processing process of each of the R feature extraction units is consistent with the data processing process of the retrieval module in the foregoing embodiment. For a related process, refer to the description in the foregoing embodiments.

As shown in FIG. 8, the electronic device respectively inputs the training sample and the Q candidate samples into the R feature extraction units. For each feature extraction unit, for example, the j^{th} feature extraction unit, the j^{th} feature extraction unit respectively extracts the object feature information of the training sample and the candidate feature information of each of the Q candidate samples. For ease of description, the object feature information of the training sample extracted by the j^{th} feature extraction unit is denoted as a j^{th} piece of sub-object feature information of the training sample. Similarly, the candidate feature information of the candidate sample extracted by the j^{th} feature extraction unit is denoted as a j^{th} piece of sub-candidate feature information of the candidate sample.

In some embodiments of this application, manners in which the j^{th} feature extraction unit extracts the j^{th} piece of sub-object feature information of the training sample and the j^{th} piece of sub-candidate feature information of the Q candidate samples include at least the following several manners:

In Manner 1, the j^{th} feature extraction unit does not consider the Q candidate samples when extracting the j^{th} piece of sub-object feature information of the training sample, and does not consider the training sample and other candidate samples among the Q candidate samples (i.e., the remaining Q−1 candidate samples) when extracting the j^{th} piece of sub-candidate feature information of each of the Q candidate samples.

In Manner 2, the j^{th} feature extraction unit considers the Q candidate samples when extracting the j^{th} piece of sub-object feature information of the training sample, and does not consider the training sample and other candidate samples among the Q candidate samples (i.e., the remaining Q−1 candidate samples) when extracting the j^{th} piece of sub-candidate feature information of each of the Q candidate samples.

A network structure of the j^{th} feature extraction unit is not limited in some embodiments of this application.

In some embodiments, as shown in FIG. 10, each feature extraction unit includes an encoder. The encoder included in the j^{th} feature extraction unit is denoted as a j^{th} encoder. Correspondingly, the extracting the j^{th} piece of sub-object feature information of the training sample (Sᵢₙₚᵤₜ) and the j^{th} piece of sub-candidate feature information of the Q candidate samples (S_{candidate}) by using the j^{th} feature extraction unit includes: performing feature extraction on the training sample by using the j^{th} encoder, to obtain the j^{th} piece of sub-object feature information of the training sample, and performing feature extraction on the Q candidate samples respectively by using the j^{th} encoder, to obtain the j^{th} piece of sub-candidate feature information of the Q candidate samples.

As shown in FIG. 10, each of the R feature extraction units in some embodiments of this application includes an encoder, and encoders included in different feature extraction units may be different. In this way, the inputted training sample and candidate samples are projected into a d-dimensional feature space through different linear projections by using different encoders respectively included in the R feature extraction units. During the retrieval, a training sample and candidate samples are encoded by using different encoders. Subsequently, candidate samples related to the training sample are identified from different perspectives, and features of the training sample are adjusted by using features of the related candidate samples, thereby enriching the features of the training sample.

A grid structure of the encoder is not limited in some embodiments of this application.

In an example, the encoder included in the j^{th} feature extraction unit may include a linear layer, and the training sample and the candidate sample are projected to a hidden dimension by using the linear layer.

S203: Select, from the Q candidate samples based on R pieces of sub-object feature information of the training sample and R pieces of sub-candidate feature information of the Q candidate samples, Kj target candidate samples satisfying a preset correlation condition with the training sample.

In some embodiments of this application, for each of the R feature extraction units, for example, the j^{th} feature extraction unit, the electronic device extracts the j^{th} piece of sub-object feature information of the training sample and the j^{th} piece of sub-candidate feature information of the candidate sample by using the j^{th} feature extraction unit. Then, Kj target candidate samples related to the training sample are selected from the Q candidate samples based on the R pieces of sub-object feature information of the training sample and the R pieces of sub-candidate feature information of the Q candidate samples. The R pieces of sub-object feature information of the training sample form the object feature information of the training sample, and the R pieces of sub-candidate feature information of the Q candidate samples form the candidate feature information of the candidate samples.

For different feature extraction units, quantities of the corresponding Kj target candidate samples may be different, and selected target candidate samples may also be different.

A manner in which the electronic device selects, from the Q candidate samples based on the R pieces of sub-object feature information of the training sample and the R pieces of sub-candidate feature information of the Q candidate samples, the Kj target candidate samples related to the training sample is not limited in some embodiments of this application.

In some embodiments, the electronic device clusters the R pieces of sub-object feature information of the training sample and the R pieces of sub-candidate feature information of the Q candidate samples, and determines candidate samples that are among the Q candidate samples and belong to the same cluster as the training sample as the Kj target candidate samples related to the training sample.

In some embodiments, S203 includes the following operations S203-A and S203-B:
S203-A: Determine a similarity between the training sample and each of the Q candidate samples based on the R pieces of sub-object feature information of the training sample and the R pieces of sub-candidate feature information of the Q candidate samples.
S203-B: Select the Kj target candidate samples from the Q candidate samples based on the similarity between the training sample and each candidate sample.

A manner in which the electronic device determines the similarity between the candidate sample and the training sample based on the R pieces of sub-candidate feature information of the candidate sample and the R pieces of sub-object feature information of the training sample is not limited in some embodiments of this application.

In a possible implementation, the electronic device calculates a distance between the R pieces of sub-object feature information of the candidate sample and the R pieces of sub-candidate feature information of the training sample by using a distance calculation manner such as a cosine distance or an L2 distance, and then determines the similarity between the candidate sample and the training sample based on the distance. In an example, a reciprocal of the distance between the R pieces of sub-candidate feature information of the candidate sample and the R pieces of sub-object feature information of the training sample may be determined as the similarity between the candidate sample and the training sample.

In some embodiments, S203-A includes the following operations S203-A1 to S203-A3:
S203-A1: Project, for a q^{th} candidate sample among the Q candidate samples, a j^{th} piece of sub-candidate feature information of the q^{th} candidate sample by using a first projection matrix, to obtain a j^{th} piece of projection feature information of the q^{th} candidate sample, q taking values 1, 2, . . . , Q.
S203-A2: Project the j^{th} piece of sub-object feature information of the training sample by using the first projection matrix, to obtain a j^{th} piece of projection feature information of the training sample.
S203-A3: Determine a distance between R pieces of projection feature information of the training sample and R pieces of projection feature information of the q^{th} candidate sample, and determine a similarity between the training sample and the q^{th} candidate sample based on the distance.

Specifically, for the q^{th} candidate sample among the Q candidate samples, the j^{th} piece of sub-candidate feature information of the q^{th} candidate sample is projected by using the first projection matrix Wₖ. For ease of description, the j^{th} piece of projected sub-candidate feature information is denoted as the j^{th} piece of projection feature information of the q^{th} candidate sample, similarly, the j^{th} piece of sub-object feature information of the training sample is projected by using the first projection matrix Wₖ, to obtain the j^{th} piece of projection feature information of the training sample. Then, the distance between the R pieces of projection feature information of the training sample and the R pieces of projection feature information of the q^{th} candidate sample is calculated, and then the distance is determined as the similarity between the training sample and the q^{th} candidate sample.

In an example, the electronic device uses the L2 distance as the similarity between the R pieces of projection feature information of the training sample and the R pieces of projection feature information of the q^{th} candidate sample. Specifically, refer to the method shown in the foregoing Formula (2).

The electronic device may determine a similarity between each of the Q candidate samples and the training sample based on the foregoing method.

Then, the electronic device selects, from the Q candidate samples based on the similarity between each of the Q candidate samples and the training sample, the Kj target candidate samples satisfying the preset correlation condition the training sample.

In an example, the electronic device selects, from the Q candidate samples, Kj target candidate samples with the greatest similarity to the training sample as the Kj target candidate samples related to the training sample.

S204: Adjust the j^{th} piece of sub-object feature information of the training sample based on a j^{th} piece of sub-candidate feature information of the Kj target candidate samples to obtain a j^{th} piece of adjusted sub-object feature information of the training sample.

In some embodiments of this application, for the j^{th} feature extraction unit among the R feature extraction units, based on the above operations, the electronic device may select, from the Q candidate samples, the Kj target candidate samples related to the training sample, and the electronic device adjusts the object feature information of the training sample based on candidate feature information of the Kj target candidate samples to obtain the adjusted object feature information of the training sample. A manner of adjustment is not limited herein.

In some embodiments, the electronic device may fuse the j^{th} piece of sub-object feature information of the training sample with the j^{th} piece of sub-candidate feature information of the Kj target candidate samples to obtain the j^{th} piece of adjusted sub-object feature information of the training sample. For example, a weight of each of the Kj target candidate samples is determined based on similarities between the Kj target candidate samples and the training sample, and then weighted processing is performed on the j^{th} piece of sub-object feature information of the training sample and the j^{th} piece of sub-candidate feature information of the Kj target candidate samples based on the weight to obtain the j^{th} piece of adjusted sub-object feature information of the training sample.

In some embodiments, S204 includes the following operations S204-A and S204-B:
S204-A: Determine, for a k^{th} target candidate sample among the Kj target candidate samples, a distance between a j^{th} piece of projection feature information of the training sample and a j^{th} piece of projection feature information of the k^{th} target candidate sample, and determine first adjustment information of the k^{th} target candidate sample for the training sample based on the distance, k taking values 1, 2, . . . , Kj.
S204-B: Adjust the j^{th} piece of sub-object feature information of the training sample based on the first adjustment information of the Kj target candidate samples for the training sample to obtain the j^{th} piece of adjusted sub-object feature information of the training sample.

In this implementation, feature adjustment information of each target candidate sample for the training sample is determined in a hidden layer by using distances between the training sample and the Kj target candidate samples. Specifically, for the k^{th} target candidate sample among the Kj target candidate samples, the distance between the j^{th} piece of projection feature information of the training sample and the j^{th} piece of projection feature information of the k^{th} target candidate sample is determined, and the first adjustment information of the k^{th} target candidate sample for the training sample is determined based on the distance.

A manner of determining the first adjustment information of the k^{th} target candidate sample for the training sample is not limited in some embodiments of this application.

In some embodiments, the electronic device may determine the distance between the j^{th} piece of projection feature information of the training sample and the j^{th} piece of projection feature information of the k^{th} target candidate sample as the first adjustment information of the k^{th} target candidate sample for the training sample.

In some embodiments, the electronic device performs activation processing on the distance between the j^{th} piece of projection feature information of the training sample and the j^{th} piece of projection feature information of the k^{th} target candidate sample, then performs dropout processing, and performs lossless linear transformation on a result of the dropout processing, to obtain second adjustment information of the k^{th} target candidate sample for the training sample. Then, the second adjustment information of the k^{th} target candidate sample for the training sample is determined as the first adjustment information of the k^{th} target candidate sample for the training sample.

Exemplarily, the electronic device determines the second adjustment information by using the foregoing Formula (3), and then determines the second adjustment information as the first adjustment information of the k^{th} candidate sample for the training sample.

In some embodiments, since the Kj target candidate samples are similar to the training sample, label information of the Kj target candidate samples may further provide additional information for features of the training sample. Based on this, in S204-A, the determining, based on the distance, first adjustment information of the k^{th} target candidate sample for the training sample includes the following operations S204-A1 and S204-A2:
S204-A1: Perform linear activation processing on the distance, then perform dropout processing, and perform lossless linear transformation on a result of the dropout processing, to obtain second adjustment information of the k^{th} target candidate sample for the training sample.
S204-A2: Determine first adjustment information of the k^{th} target candidate sample for the training sample based on the second adjustment information of the k^{th} target candidate sample for the training sample and label information of the k^{th} target candidate sample.

For a process of performing linear activation processing on the distance between the j^{th} piece of projection feature information of the training sample and the j^{th} piece of projection feature information of the k^{th} target candidate sample, then performing dropout processing, and performing lossless linear transformation on the result of the dropout processing, to obtain the second adjustment information of the k^{th} target candidate sample for the training sample, refer to the description in the foregoing embodiments. Details are not described herein again.

Then, the electronic device determines the first adjustment information of the k^{th} target candidate sample for the training sample based on the second adjustment information of the k^{th} target candidate sample for the training sample and the label information of the k^{th} target candidate sample.

In a possible implementation, the electronic device determines a sum of the second adjustment information of the k^{th} target candidate sample for the training sample and the label information of the k^{th} target candidate sample as the first adjustment information of the k^{th} target candidate sample for the training sample.

In a possible implementation, the label information of the k^{th} target candidate sample is projected by using a second projection matrix, to obtain label projection information of the k^{th} target candidate sample; and a sum of the second adjustment information of the k^{th} target candidate sample for the training sample and the label projection information of the k^{th} target candidate sample is determined as the first adjustment information of the k^{th} target candidate sample for the training sample.

A process in which the electronic device determines the first adjustment information of the k^{th} target candidate sample among the Kj target candidate samples for the training sample is described above. With reference to the above operations, the electronic device may determine first adjustment information of each of the Kj target candidate samples for the training sample, and then perform the above operation S204-B to adjust the j^{th} piece of sub-object feature information of the training sample based on the first adjustment information of the Kj target candidate samples for the training sample and determine the j^{th} piece of adjusted sub-object feature information of the training sample, and R pieces of adjusted sub-object feature information of the training sample form the adjusted object feature information of the training sample.

A manner of adjusting the j^{th} piece of sub-object feature information of the training sample based on the first adjustment information of the Kj target candidate samples for the training sample and determining the j^{th} piece of adjusted sub-object feature information of the training sample is not limited in some embodiments of this application.

In some embodiments, the electronic device adds a sum or an average value of the first adjustment information of the Kj target candidate samples for the training sample to the j^{th} piece of sub-object feature information of the training sample to obtain the j^{th} piece of adjusted sub-object feature information of the training sample.

In some embodiments, S204-B includes the following operations S204-B1 to S204-B3:
S204-B1: Determine, for the k^{th} target candidate sample among the Kj target candidate samples, a weight of the k^{th} target candidate sample based on a similarity between the training sample and the k^{th} target candidate sample.
S204-B2: Perform, based on weights of the Kj target candidate samples, weighted processing on the first adjustment information of the Kj target candidate samples for the training sample to obtain weighted adjustment information.
S204-B3: Determine the j^{th} piece of adjusted sub-object feature information of the training sample based on the j^{th} piece of sub-object feature information of the training sample and the weighted adjustment information.

In this implementation, for each of the Kj target candidate samples, for example, the k^{th} target candidate sample, the weight of the k^{th} target candidate sample is determined based on a similarity between the k^{th} target candidate sample and the training sample. The weight is positively correlated with the similarity.

A manner in which the electronic device determines the weight of the k^{th} target candidate sample based on the similarity sim between the k^{th} target candidate sample and the training sample is not limited in some embodiments of this application. For example, the similarity sim between the k^{th} target candidate sample and the training sample is determined as the weight of the k^{th} target candidate sample.

In a possible implementation, normalization processing is performed on the similarity sim between the k^{th} target candidate sample and the training sample to obtain the weight of the k^{th} target candidate sample. Exemplarily, a weight of each of the Kj target candidate samples may be determined by using the method shown in Formula (5).

Based on the above operations, the electronic device may determine the weight of each of the at least one target candidate sample, and perform, based on the weights of the Kj target candidate samples, weighted processing on the first adjustment information of the Kj target candidate samples for the training sample to obtain weighted adjustment information. Then, the j^{th} piece of adjusted sub-object feature information of the training sample is determined based on the j^{th} piece of sub-object feature information of the training sample and the weighted adjustment information. For example, a sum of the j^{th} piece of sub-object feature information of the training sample and the weighted adjustment information is determined as the j^{th} piece of adjusted sub-object feature information of the training sample.

The j^{th} feature extraction unit among the R feature extraction units is taken as an example above to describe a process of determining, by using the j^{th} feature extraction unit, the j^{th} piece of adjusted sub-object feature information of the training sample. With reference to the foregoing method, the electronic device may determine adjustment information of each of the R feature extraction units for the training sample, to obtain R pieces of adjusted sub-object feature information of the training sample, which are denoted as h₁, h₂, . . . , hⱼ, . . . , and hᵣ, where hⱼ denotes the j^{th} piece of adjusted sub-object feature information of the training sample corresponding to the j^{th} feature extraction unit.

In some embodiments, the electronic device may compare and analyze the R pieces of sub-object feature information of the training sample and the R pieces of sub-candidate feature information of the Kj target candidate samples, for example, calculate a variance between the j^{th} piece of sub-object feature information and the j^{th} piece of sub-candidate feature information of the Kj target candidate samples, and if a variance corresponding to the j^{th} piece of sub-object feature information is higher than a threshold, delete the j^{th} piece of sub-object feature information, to obtain the adjusted object feature information of the training sample. In another example, if the variance corresponding to the j^{th} piece of sub-object feature information is higher than the threshold, it is determined that the j^{th} piece of sub-object feature information is modified. For example, a mean or a weighted mean of the j^{th} piece of sub-candidate feature information of the Kj target candidate samples is calculated, and the j^{th} piece of sub-object feature information is modified into the mean or the weighted mean.

S205: Classify, by using a classification module, R pieces of adjusted sub-object feature information of the training sample, to obtain a classification result of the training sample.

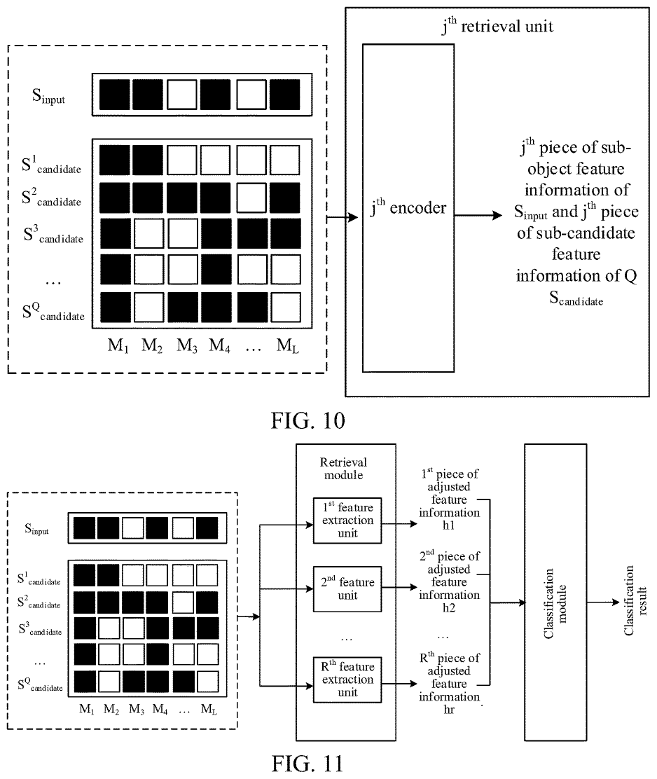

As shown in FIG. 11, in some embodiments of this application, the electronic device classifies, by using the classification module, the R pieces of adjusted sub-object feature information of the training sample, to obtain a classification result of the training sample.

In some embodiments, the electronic device fuses the R pieces of adjusted sub-object feature information of the training sample (processing such as addition, concatenation, or multiplication), and classifies fused feature information by using the classification module, to obtain the classification result of the training sample.

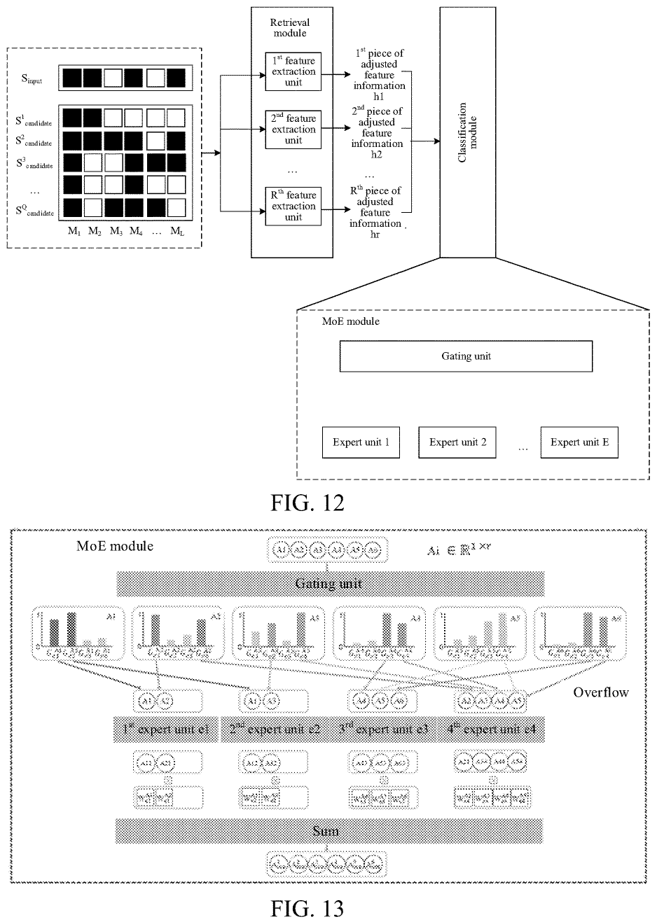

In some embodiments, as shown in FIG. 12, the classification module includes a MoE module, and the MoE module includes a gating unit and E expert units. The gating unit is configured to determine that each input token is assigned to the expert units with top-n probabilities. In this case, S205 includes the following operations S205-A to S205-C:

S205-A: Obtain S input tokens based on the R pieces of adjusted sub-object feature information of the training sample, S being a positive integer greater than 1.

In some embodiments of this application, for the j^{th} feature extraction unit among the R feature extraction units, the j^{th} piece of adjusted sub-object feature information of the training sample corresponding to the j^{th} feature extraction unit includes adjusted feature information of the L biological state parameters, and the j^{th} piece of adjusted sub-object feature information includes d elements, that is, a vector with a length of d. In this way, the R pieces of adjusted sub-object feature information of the training sample may form a matrix.

In some embodiments, each of the R pieces of adjusted sub-object feature information of the training sample is taken as a row of a first matrix, to obtain the first matrix including R rows; and each column of the first matrix is taken as one input token, to obtain the S input tokens. If a dimension of each piece of adjusted sub-object feature information is d, S is equal to d.

Exemplarily, the first matrix H may be represented as: H=Concat (h₁, h₂, . . . , hᵣ)^{T}, where H∈R^{d×r}, Cancat represents a concatenation operation, and each element in the d dimensions may be regarded as an input token (also referred to as a mark) in a subsequent MoE module. Exemplarily, in some embodiments of this application, the input token is represented with the letter A. r dimensions are embedded as a hidden layer thereof.

S205-B: For an s^{th} input token among the S input tokens, determine, by using the gating unit, a probability value indicating that the s^{th} input token is assigned to each of the E expert units.

E is a positive integer greater than 1, and s takes values 1, 2, . . . , S.

Exemplarily, the electronic device may determine, by using the following Formula (7), the probability value indicating that each input token A is assigned to each expert unit:

G=H·W_{Gate} (7)

where W_{Gate} denotes a learnable parameter matrix, and G denotes a probability value indicating that an input token is assigned to each expert unit. H denotes the first matrix.

Exemplarily, as shown in FIG. 13, FIG. 13 is based on an example in which a quantity S of input tokens is equal to 6 and a quantity E of expert units is equal to 4. The electronic device determines, by using the gating unit, probability values indicating that the 6 input tokens A1, A2, A3, A4, A5, and A6 are assigned to each of the 4 expert units. For example, the probability values indicating that the input token A1 is assigned to the 4 expert units are sequentially

$${G}_{e 1}^{A 1} , {G}_{e 2}^{A 2} , {G}_{e 3}^{A 3} , and {G}_{e 4}^{A 4} , where {G}_{e 1}^{A 1}$$

denotes the probability value indicating that the input token A1 is assigned to the 1^{st} expert unit e1 for processing, and by analogy,

$${G}_{e 4}^{A 4}$$

denotes the probability value indicating that the input token A1 is assigned to the 4^{th} expert unit e4 for processing.

S205-C: Select, from the E expert units based on the probability values, T expert units configured to process the s^{th} input token, and respectively process the s^{th} input token by using the T expert units, to obtain fused feature information of the s^{th} input token.

T is a positive integer less than E.

Based on the above operations, for the s^{th} input token among the S input tokens, the electronic device may determine a probability value indicating that the s^{th} input token is assigned to each of the E expert units. Then, the T expert units configured to process the s^{th} input token are selected from the E expert units according to the probability values. That is, T expert units with maximum probability values are selected from the E expert units.

Exemplarily, the electronic device may determine, by using the following Formula (8), the T expert units configured to process the s^{th} input token:

topT_indicesₛ=topT(Gₛ),∀∈{1,2, . . . ,d} (8)

where Gₛ denotes the probability values indicating that the s^{th} input token is assigned to the E expert units, and topN(·) represent a position of an N^{th} element with the highest probability after each input token (d) in an expert dimension passes through W_{Gate}. The T top expert units may receive the s^{th} input token. topT_indices, denotes the T expert units configured to process the s^{th} input token.

Still referring to FIG. 13, in an example in which the s^{th} input token is the 1^{st} input token A1, as shown in FIG. 13, among the probability values indicating that the 1^{st} input token A1 is assigned to the 4 expert units, the probability value

$${G}_{e 1}^{A 1}$$

indicating assignment to the first expert unit e1 and the probability value

$${G}_{e 2}^{A 2}$$

indicating assignment to the second expert unit e2 are the highest. Therefore, it may be determined that the 1^{st} input token A1 is assigned to the 1st expert unit e1 and the 2^{nd} expert unit e2 for processing. In this case, T is equal to 2.

In an actual operation, some expert units may receive a large quantity of input tokens, while other experts receive only a small quantity of input tokens. This phenomenon is referred to as an unbalanced expert load. This situation not only contradicts an initial design intention of the data classification model in some embodiments of this application, but also affects computational efficiency (for example, causes uneven communication load in distributed training). Based on this, in some embodiments of this application, a fixed capacity value C is set for each expert unit (that is, a maximum quantity of marks that can be accepted by each expert unit). If a quantity of input tokens received by an expert unit exceeds a capacity thereof, the expert unit no longer receives any input token. In this case, excess input tokens are referred to as overflow.

Based on the above operations, the electronic device, after selecting, from the E expert units, the T expert units configured to process the s^{th} input token, assign the s^{th} input token to the T expert units for processing respectively, to obtain fused feature information of the s^{th} input token.

A manner in which the electronic device respectively processes the s^{th} input token by using the T expert units, to obtain the fused feature information of the s^{th} input token is not limited in some embodiments of this application.

In some embodiments, the electronic device respectively inputs the s^{th} input token into the T expert units, to obtain feature information obtained by respectively processing the s^{th} input token by the T expert units, and then may obtain T pieces of feature information. Then, the T pieces of feature information are fused. For example, the T pieces of feature information are added to obtain the fused feature information of the s^{th} input token, or an average value of the T pieces of feature information is determined as the fused feature information of the s^{th} input token.

In some embodiments, in S205-C, the respectively processing the s^{th} input token by using the T expert units, to obtain fused feature information of the s^{th} input token includes the following operations S205-C1 to S205-C3:
S205-C1: For each of the T expert units, perform, by using the expert unit, feature fusion processing on input tokens assigned to the expert unit, to obtain output feature information of the input tokens, the input tokens including the s^{th} input token.
S205-C2: Determine, based on the probability value indicating that the s^{th} input token is assigned to each of the T expert units, weights respectively corresponding to the T expert units.
S205-C3: Perform, based on the weights respectively corresponding to the T expert units, weighted processing on the output feature information of the s^{th} input token respectively outputted by the T expert units, to obtain the fused feature information of the s^{th} input token.

In this implementation, for each of the T expert units, the electronic device performs, by using the expert unit, feature fusion processing on input tokens assigned to the expert unit, to obtain output feature information of the input tokens, where the input tokens include the s^{th} input token.

A network structure of the expert unit is not limited in some embodiments of this application. In an example, the expert unit includes two linear transformations and an activation function, for example, an ReLU activation function or an activation function of another type.

In some embodiments, in the E expert units included in the data classification model in some embodiments of this application, each expert unit has a unified structure instead of a different structure designed for some classifications (for example, cancer types), so that the data classification model provided in some embodiments of this application may be extended to other data sets.

In some embodiments of this application, the electronic device further determines, based on the probability value indicating that the s^{th} input token is assigned to each of the T expert units, weights respectively corresponding to the T expert units.

Exemplarily, the electronic device determines, by using the following Formula (9), the weights respectively corresponding to the expert units:

W=softmax(TopT(G)) (9)

In this example, the electronic device uses a softmax function to normalize the probability values indicating that the s^{th} input token is assigned to the T expert units, and may obtain a weight of each input token assigned to the T expert units.

Then, based on the weights respectively corresponding to the T expert units, weighted processing is performed on the output feature information of the s^{th} input token respectively outputted by the T expert units, to obtain the fused feature information of the s^{th} input token.

The s^{th} input token among the S input tokens is taken as an example above to describe a process of determining the fused feature information of the s^{th} input token. With reference to the method, the electronic device may determine fused feature information of each of the S input tokens.

Exemplarily, the electronic device may determine the fused feature information

$${A}_{MOE}^{S}$$

of the s^{th} input token by using the following Formula (10):

$${A}_{MOE}^{S} = Ws \cdot {E}_{MOE}^{S} \qquad ( 10 )$$

where

$${A}_{MOE}^{S} ∈ {R}^{d \times r} ,$$

with a shape the same as that of the first matrix H, and

$${E}_{MOE}^{S}$$

denotes the output feature information of the s^{th} input token outputted by the expert unit.

Still referring to FIG. 13, in an example in which the s^{th} input token is the 1^{st} input token A1, the 1^{st} input token is assigned to the 1^{st} expert unit e1 and the 2^{nd} expert unit e2, where a weight for the 1^{st} expert unit e1 to process the 1^{st} input token A1 is

$${W}_{e 1}^{A 1} ,$$

and a weight for the 2^{nd} expert unit e2 to process the 1^{st} input token A1 is

$${W}_{e 2}^{A 1} .$$

In this way, a weight matrix corresponding to the 1^{st} input token A1 is

$$W 1 = ( {W}_{e 1}^{A 1} , {W}_{e 2}^{A 1} ) .$$

The 1^{st} expert unit processes the 1^{st} input token A1, and obtained output feature information is A11. The 2^{nd} expert unit processes the 1^{st} input token A1, and obtained output feature information is A12. In this case, output feature information corresponding to the 1^{st} input token A1 is

$${E}_{MOE}^{S} = ( A 11 , A 12 ) .$$

In this way, fused feature information of the 1^{st} input token A1 is

$${A}_{MOE}^{S} = {W}_{e 1}^{A 1} * A 11 + {W}_{e 2}^{A 1} * A 12.$$

With reference to the foregoing method, the electronic device may determine fused feature information of each of the S input tokens. Then, the following operation S205-D is performed.

S205-D: Obtain mixed feature information of the training sample based on the fused feature information of the S input tokens.

A manner in which the electronic device obtains the mixed feature information of the training sample based on the fused feature information of the S input tokens is not limited in some embodiments of this application.

In some embodiments, an average value or a sum of the fused feature information of the S input tokens is determined as the mixed feature information of the training sample.

In some embodiments, the fused feature information of the S input tokens is concatenated to obtain the mixed feature information of the training sample.

S205-E: Classify the training sample based on the mixed feature information to obtain a classification result of the training sample.

A manner in which the electronic device classifies the training sample based on the mixed feature information to obtain the classification result of the training sample is not limited in some embodiments of this application.

In some embodiments, the mixed feature information of the training sample is classified to obtain the classification result of the training sample.

In some embodiments, linear transformation is performed on the mixed feature information of the training sample by using a linear layer, to reduce a dimension and aggregate features. Subsequently, the hidden layer is mapped to a final output dimension by using a simple linear classification head. Then, the probabilities are normalized to generate a final output, and probability values indicating that the training sample belongs to preset classifications are predicted, to obtain the classification result of the training sample.

Exemplarily, the electronic device may determine the adjusted classification result of the training sample by using the following Formula (11):

P_{y}=softmax(FC_{head}(Linear(h_{MOE}))) (11)

where p_{y} denotes the classification result of the training sample, that is, the probability values indicating that the training sample belongs to the preset classifications, Linear denotes the linear layer, FC_{head} denotes the linear classification head, and Softmax denotes a normalization function.

S206: Determine a loss of the data classification model based on the classification result of the training sample, and train the data classification model based on the loss.

In some embodiments, the electronic device determines a first loss based on the classification result of the training sample and label information of the training sample, and then determines the first loss as the loss of the data classification model.

Exemplarily, the electronic device determines the first loss Lₘₐᵢₙ(p_{y},y) by using the following Formula (12):

$${L}_{main} ( {p}_{y} , y ) = - {\sum }_{i = 1}^{{n}_{class}} {y}_{i} * loy ( {p}_{{y}_{i}} ) \qquad ( 12 )$$

where n_{class} denotes a quantity of preset classifications, yᵢ denotes a true probability that the training sample belongs to an i^{th} classification in the preset classifications, and p_{y}, denotes a probability value indicating that the training sample belongs to the i^{th} classification and predicted by the data classification model.

In addition to calculating the first loss by using a classification cross-entropy loss function Lₘₐᵢₙ, the electronic device may calculate the first loss by using another loss function, which is not limited in some embodiments of this application.

In some embodiments, S206 includes the following operations S206-A to S206-C:
S206-A: Determine a first loss based on the classification result of the training sample and label information of the training sample.
S206-B: Determine a second loss based on a quantity of input labels assigned to each of the E expert units and an assignment probability value.
S206-C: Determine the loss of the data classification model based on the first loss and the second loss.

In this implementation, to implement even distribution among the expert units, prevent unbalanced assignment of the input tokens, and simultaneously avoid discarding of excessive input tokens due to overlapping, a balance loss function L_{balance} is introduced in some embodiments of this application. The balance loss function aims to minimize a squared difference of a proportion of input tokens cₑ/S received by each expert unit. However, cₑ is obtained through a top-T process, which is not differentiable. To resolve this problem, in some embodiments of this application, an average probability mₑ of all tokens received by each expert unit is used as a differentiable alternative, to replace the square with mₑ(cₑ/S). In this way, in some embodiments of this application, the loss function may be optimized by using gradient descent, thereby effectively preserving an original concept, resolving a problem of non-differentiation, and ensuring balanced distribution of the input tokens assigned to the expert units.

Exemplarily, the electronic device may determine the second loss L_{balance} by using the following Formula (13):

$${L}_{balance} = \frac{1}{E} {\sum }_{e = 1}^{E} \frac{{c}_{e}}{S} {m}_{e} \qquad ( 13 )$$

where E denote a quantity of expert units, cₑ denotes a quantity of input tokens assigned to an e^{th} expert unit among the E expert units, S denotes a total quantity of the input tokens, and mₑ denotes an average probability of all tokens assigned to the e^{th} expert unit.

The electronic device, after determining the first loss and the second loss based on the above operations, determines the loss of the data classification model based on the first loss and the second loss.

In an example, the electronic device determines a sum of the first loss and the second loss as the loss of the data classification model.

In an example, the electronic device multiplies the second loss by a balance coefficient, which is then added to the first loss, to obtain the loss of the data classification model.

Exemplarily, the electronic device determines the loss L of the data classification model according to the following Formula (14):

L=Lₘₐᵢₙ+α*L_{balance} (14)

where α denotes the balance coefficient.

Then, the electronic device adjusts parameters in the data classification model based on the foregoing loss. In this way, multiple rounds of training are performed, to obtain a trained data classification model.

In the model training method provided in some embodiments of this application, the data classification model includes a retrieval module and a classification module, and the data classification model includes R feature extraction units. R pieces of sub-object feature information of the training sample and R pieces of sub-candidate feature information of each of the Q candidate samples are extracted by using the R feature extraction units. For each feature extraction unit, for example, a j^{th} feature extraction unit, Kj target candidate samples satisfying the preset correlation condition with the training sample are selected from the Q candidate samples based on a j^{th} piece of sub-object feature information of the training sample and a j^{th} piece of sub-candidate feature information of the Q candidate samples. Then, the j^{th} piece of sub-object feature information of the training sample is adjusted based on a j^{th} piece of sub-candidate feature information of the Kj target candidate samples to obtain a j^{th} piece of adjusted sub-object feature information of the training sample. By analogy, R pieces of adjusted sub-object feature information of the training sample may be obtained. Then, the R pieces of adjusted sub-object feature information of the training sample are classified by using the classification module in the data classification model, to obtain a classification result of the training sample. For example, the R pieces of adjusted sub-object feature information of the training sample are mixed by using a MoE module, and then data classification is performed, to obtain the classification result of the training sample. Then, a loss of the data classification model is determined based on the classification result of the training sample, and the data classification model is trained based on the loss. As can be known, features of similar objects are extracted multiple times from different linear transformation spaces, so that the R pieces of adjusted sub-object feature information of the training sample include richer features. Further, when data classification is performed by using the R pieces of adjusted sub-object feature information of the training sample, multiple different classifications may be covered. For example, classification reference results of a plurality of different types of early-stage cancers may be obtained, and classification accuracy may be improved.

A training process of the data classification model is described above, and a data classification process performed based on the trained data classification model is described below.

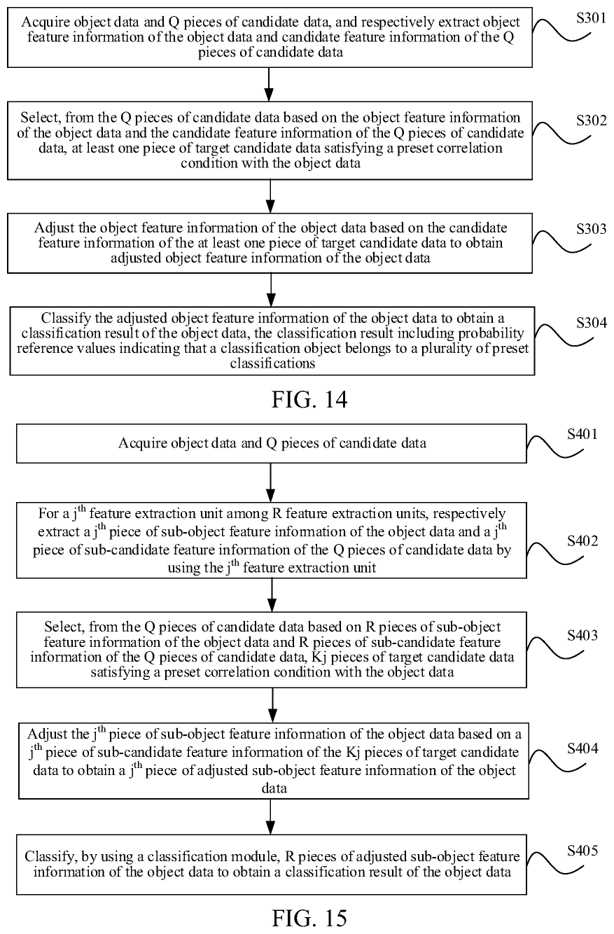

FIG. 14 is a schematic flowchart of a data classification method according to some embodiments. Some embodiments of this application may be performed by an apparatus having a data classification function, for example, a data classification apparatus. The data classification apparatus may be an electronic device such as the server 102 or the terminal device 101 shown in FIG. 1, or may be a system including the server 102 or 101 shown in FIG. 1. For ease of description, the method in some embodiments of this application is described in the following embodiment by using an electronic device as an example.

As shown in FIG. 14, the data classification method in some embodiments of this application includes the following operations:

S301: Acquire object data and Q pieces of candidate data, and respectively extract object feature information of the object data and candidate feature information of the Q pieces of candidate data.

The object data includes parameter values of L biological state parameters of a classification object. The candidate data includes parameter values of L biological state parameters of a reference object different from the classification object. Both Q and L are positive integers greater than 1.

In some embodiments of this application, a candidate data set is involved, and the candidate data set includes a plurality of different pieces of candidate data. When the object data may be classified, the electronic device may acquire, from the candidate data set, Q pieces of candidate data corresponding to the object data. For example, the Q pieces of candidate data are randomly selected from the candidate data set.

A manner in which the electronic device extracts the object feature information of the object data and the candidate feature information of the Q pieces of candidate data is not limited in some embodiments of this application. For example, the object feature information of the object data and the candidate feature information of the Q pieces of candidate data are extracted by using a trained feature extraction model.

In some embodiments, the electronic device extracts the object feature information of the object data and the candidate feature information of the Q pieces of candidate data by using the retrieval module in the foregoing data classification model.

A implementation process of S301 above is basically consistent with the implementation process of S101 above. Refer to the related description of S101 above. Details are not described herein again.

S302: Select, from the Q pieces of candidate data based on the object feature information of the object data and the candidate feature information of the Q pieces of candidate data, at least one piece of target candidate data satisfying a preset correlation condition with the object data.

A implementation process of S302 above is basically consistent with the implementation process of S102 above. Refer to the related description of S102 above. Details are not described herein again.

S303: Adjust the object feature information of the object data based on the candidate feature information of the at least one piece of target candidate data to obtain adjusted object feature information of the object data.

In some embodiments of this application, adjusting the object feature information of the object data may be understood as expanding the object feature information of the object data to enrich the object feature information of the object data, so that the adjusted object feature information includes not only feature information of a classification object but also feature information of a similar reference object.

A implementation process of S303 above is basically consistent with the implementation process of S103 above. Refer to the related description of S103 above. Details are not described herein again.

S304: Classify the adjusted object feature information of the object data to obtain a classification result of the object data, the classification result including probability reference values indicating that a classification object belongs to a plurality of preset classifications.

A method in which the electronic device classifies the adjusted object feature information of the object data to obtain the classification result of the object data is not limited in some embodiments of this application. For example, the adjusted object feature information of the object data is classified by using a trained data classification model, to obtain the classification result of the object data.

In some embodiments, the electronic device classifies the adjusted object feature information of the object data by using the classification module in the foregoing data classification model, to obtain the classification result of the object data.

If the data classification method in some embodiments of this application is applied to disease diagnosis, the classification result obtained in some embodiments of this application is a disease classification reference result, not a direct result of disease diagnosis. For example, when the data classification method in some embodiments of this application is applied to detection of early-stage cancers, a classification reference result indicating whether the classification object belongs to a plurality of types of early-stage cancers may be obtained in some embodiments of this application.

A implementation process of S304 above is basically consistent with the implementation process of S104 above. Refer to the related description of S104 above. Details are not described herein again.

In the data classification method in some embodiments of this application, during data classification, object data and Q pieces of candidate data are first acquired. Object feature information of the object data and candidate feature information of the Q pieces of candidate data are respectively extracted. The object data includes parameter values of L biological state parameters of a classification object. The candidate data includes parameter values of L biological state parameters of a reference object different from the classification object. Then, at least one piece of target candidate data satisfying a preset correlation condition with the object data is selected from the Q pieces of candidate data based on the object feature information of the object data and the candidate feature information of the Q pieces of candidate data, and the object feature information is adjusted based on the candidate feature information of the at least one piece of target candidate data, to obtain adjusted object feature information. Finally, the adjusted object feature information of the object data is classified to obtain a classification result of the object data. The classification result includes probability reference values indicating that the classification object belongs to a plurality of preset classifications. In some embodiments of this application, the at least one target candidate data selected from the Q pieces of candidate data is data of another object similar to the classification object. The object feature information of the object data is adjusted by using the candidate feature information of the at least one piece of target candidate data, so that features of similar objects can be integrated, making the adjusted object feature information of the object data richer and more comprehensive. Further, when the adjusted object feature information of the object data is used for data classification, a plurality of different classifications may be covered. For example, classification reference results of a plurality of different types of early-stage cancers can be obtained, and classification accuracy can be improved.

The data classification process involved in some embodiments of this application is described hereinabove. In some embodiments, as shown in FIG. 8, the retrieval module in some embodiments of this application includes R feature extraction units.

FIG. 15 is a schematic flowchart of a data classification method according to some embodiments. As shown in FIG. 15, a data detection process in some embodiments of this application includes the following operations:

S401: Acquire object data and Q pieces of candidate data.

A implementation process of S401 above is consistent with that of S301 above. Refer to the related description of S301 above. Details are not described herein again.

S402: For a j^{th} feature extraction unit among R feature extraction units, respectively extract a j^{th} piece of sub-object feature information of the object data and a j^{th} piece of sub-candidate feature information of the Q pieces of candidate data by using the j^{th} feature extraction unit.

j takes values 1, 2, . . . , R. The object feature information of the object data includes R pieces of sub-object feature information, and the candidate feature information of each piece of candidate data includes R pieces of sub-candidate feature information.

For example, the feature extraction units may be encoders. The j^{th} piece of sub-object feature information of the object data and the j^{th} piece of sub-candidate feature information of the Q pieces of candidate data are respectively extracted by using a j^{th} encoder.

For a implementation process of S402 above, refer to the related description of S202 above. Details are not described herein again.

S403: Select, from the Q pieces of candidate data based on R pieces of sub-object feature information of the object data and R pieces of sub-candidate feature information of the Q pieces of candidate data, Kj pieces of target candidate data satisfying a preset correlation condition with the object data.

For example, a similarity between the object data and each of the Q pieces of candidate data is determined based on the R pieces of sub-object feature information of the object data and the R pieces of sub-candidate feature information of the Q pieces of candidate data; and the Kj pieces of target candidate data are selected from the Q pieces of candidate data based on the similarity.

In some embodiments, the determining a similarity between the object data and each of the Q pieces of candidate data based on the R pieces of sub-object feature information of the object data and the R pieces of sub-candidate feature information of the Q pieces of candidate data includes: projecting, for a q^{th} piece of candidate data among the Q pieces of candidate data, a j^{th} piece of sub-candidate feature information of the q^{th} piece of candidate data by using a first projection matrix, to obtain a j^{th} piece of projection feature information of the q^{th} piece of candidate data, q taking values 1, 2, . . . , Q; projecting the j^{th} piece of sub-object feature information of the object data by using the first projection matrix, to obtain the j^{th} piece of projection feature information of the object data; and determining a distance between R pieces of projection feature information of the object data and R pieces of projection feature information of the q^{th} piece of candidate data, and determining a similarity between the object data and the q^{th} piece of candidate data based on the distance.

For a implementation process of S403 above, refer to the related description of S203 above. Details are not described herein again.

S404: Adjust the j^{th} piece of sub-object feature information of the object data based on a j^{th} piece of sub-candidate feature information of the Kj pieces of target candidate data to obtain a j^{th} piece of adjusted sub-object feature information of the object data.

For example, for a k^{th} piece of target candidate data among the Kj pieces of target candidate data, a distance between the j^{th} piece of projection feature information of the object data and a j^{th} piece of projection feature information of the k^{th} piece of target candidate data is determined, and first adjustment information of the k^{th} piece of target candidate data for the object data is determined based on the distance, k taking values 1, 2, . . . , Kj; and the j^{th} piece of sub-object feature information of the object data is adjusted based on respective first adjustment information of the Kj pieces of target candidate data for the object data, and the j^{th} piece of adjusted sub-object feature information of the object data is determined.

In some embodiments, the determining, based on the distance, first adjustment information of the k^{th} piece of target candidate data for the object data includes: performing linear activation processing on the distance, then performing dropout processing, and performing lossless linear transformation on a result of the dropout processing, to obtain second adjustment information of the k^{th} piece of target candidate data for the object data; and determining the first adjustment information of the k^{th} piece of target candidate data for the object data based on the second adjustment information of the k^{th} piece of target candidate data for the object data and label information of the k^{th} piece of target candidate data.

In some embodiments, the determining the first adjustment information of the k^{th} piece of target candidate data for the object data based on the second adjustment information of the k^{th} piece of target candidate data for the object data and label information of the k^{th} piece of target candidate data includes: projecting the label information of the k^{th} piece of target candidate data by using a second projection matrix, to obtain label projection information of the k^{th} piece of target candidate data; and determining a sum of the second adjustment information of the k^{th} piece of target candidate data for the object data and the label projection information of the k^{th} piece of target candidate data as the first adjustment information of the k^{th} piece of target candidate data for the object data.

In some embodiments, the adjusting the j^{th} piece of sub-object feature information of the object data based on respective first adjustment information of the Kj pieces of target candidate data for the object data, and determining the j^{th} piece of adjusted sub-object feature information of the object data includes: determining, for a k^{th} piece of target candidate data among the Kj pieces of target candidate data, a weight of the k^{th} piece of target candidate data based on a similarity between the object data and the k^{th} piece of target candidate data; performing, based on weights of the Kj pieces of target candidate data, weighted processing on the first adjustment information of the Kj pieces of target candidate data for the object data, to obtain weighted adjustment information; and determining, based on the j^{th} piece of sub-object feature information of the object data and the weighted adjustment information, the j^{th} piece of adjusted sub-object feature information of the object data.

For a implementation process of S404 above, refer to the related description of S204 above. Details are not described herein again.

S405: Classify, by using a classification module, R pieces of adjusted sub-object feature information of the object data to obtain a classification result of the object data.

In some embodiments, the classification module includes a MoE module, the MoE module includes a gating unit and E expert units, and the classifying, by using a classification module, R pieces of adjusted sub-object feature information of the object data to obtain a classification result of the object data includes:
obtaining S input tokens based on the R pieces of adjusted sub-object feature information of the object data, S being a positive integer greater than 1. For example, each of the R pieces of adjusted sub-object feature information of the object data is taken as a row of a first matrix, to obtain the first matrix including R rows; and each column of the first matrix is taken as one input token, to obtain the S input tokens.

Then, for an s^{th} input token among the S input tokens, a probability value indicating that the s^{th} input token is assigned to each of the E expert units is determined by using the gating unit, E being a positive integer greater than 1, and S being a positive integer less than or equal to S; and based on the probability values, T expert units configured to process the s^{th} input token are selected from the E expert units, and the s^{th} input token is processed respectively by using the T expert units, to obtain fused feature information of the s^{th} input token, T being a positive integer less than E. In some embodiments, for each of the T expert units, by using the expert unit, feature fusion processing is performed on input tokens assigned to the expert unit, to obtain output feature information of the input tokens, the input tokens including the s^{th} input token; weights respectively corresponding to the T expert units are determined based on the probability value indicating that the s^{th} input token is assigned to each of the E expert units; and based on the weights respectively corresponding to the T expert units, weighted processing is performed on output feature information of the s^{th} input token respectively outputted by the T expert units, to obtain the fused feature information of the s^{th} input token.

Then, mixed feature information of the object data is obtained based on the fused feature information of the S input tokens; and the object data is classified based on the mixed feature information to obtain a classification result of the object data.

For a implementation process of S405 above, refer to the related description of S205 above. Details are not described herein again.

In some embodiments of this application, extraction is performed multiple times from each piece of candidate data to extract R pieces of sub-candidate feature information, and then features of the object data are adjusted based on extracted R sub-candidate features, to achieve different adjustments to the features of the object data in different linear spaces, thereby enriching feature information of the object data. In this way, when data classification is performed based on the rich feature information of the object data, accuracy of classification of the object data can be improved.

The data classification process in some embodiments of this application is described below with reference to FIG. 14 based on an example in which the L biological state parameters include L gene mutations and the classification result includes probability reference values indicating that the object data belongs to a plurality of cancer classifications.

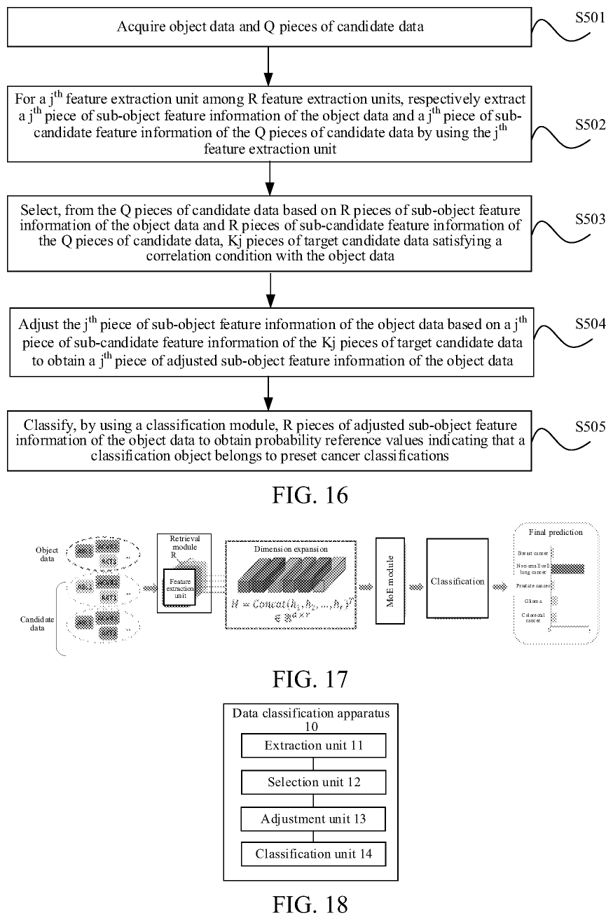

FIG. 16 is a schematic flowchart of a data classification method according to some embodiments. As shown in FIG. 16, the data detection method in some embodiments of this application includes the following operations:

S501: Acquire object data and Q pieces of candidate data.

The object data includes state values of L gene mutations of a classification object. The candidate data includes state values of L gene mutations of another reference object different from the classification object. Both Q and L are positive integers greater than 1.

Exemplarily, as shown in FIG. 17, the first dotted circle includes state values of L gene mutations of the classification object, where the L gene mutations include ABL1, ACVR1, AKT1, and the like. The remaining dotted circles include state values of L gene mutations of other reference objects.

S502: For a j^{th} feature extraction unit among R feature extraction units, respectively extract a j^{th} piece of sub-object feature information of the object data and a j^{th} piece of sub-candidate feature information of the Q pieces of candidate data by using the j^{th} feature extraction unit.

For a implementation process of S502 above, refer to the related description of S202 above. Details are not described herein again.

S503: Select, from the Q pieces of candidate data based on R pieces of sub-object feature information of the object data and R pieces of sub-candidate feature information of the Q pieces of candidate data, Kj pieces of target candidate data satisfying a correlation condition with the object data.

For a implementation process of S503 above, refer to the related description of S203 above. Details are not described herein again.

S504: Adjust the j^{th} piece of sub-object feature information of the object data based on a j^{th} piece of sub-candidate feature information of the Kj pieces of target candidate data to obtain a j^{th} piece of adjusted sub-object feature information of the object data.

As shown in FIG. 17, the electronic device respectively inputs the object data and the Q pieces of candidate data into the R feature extraction units, and each feature extraction unit processes the object data and the Q pieces of candidate data, to finally obtain R pieces of adjusted sub-object feature information of the object data, which are, for example, h₁, h₂, . . . , and hᵣ. The R pieces of adjusted sub-object feature information are concatenated to obtain a first matrix H.

For a implementation process of S504 above, refer to the related description of S204 above. Details are not described herein again.

S505: Classify, by using a classification module, R pieces of adjusted sub-object feature information of the object data to obtain probability reference values indicating that a classification object belongs to preset cancer classifications.

In some embodiments of this application, the probability reference values indicating that the classification object belongs to the preset cancer classifications may be understood as a classification reference result indicating that the classification object belongs to the preset cancer classifications, so that relevant personnel diagnose the classification object based on the classification reference result.

In some embodiments, as shown in FIG. 17, the classification module includes a MoE module, the MoE module includes a gating unit and E expert units, and the classifying, by using a classification module, R pieces of adjusted sub-object feature information of the object data under the R feature extraction units respectively to obtain probability reference values indicating that the object data belongs to preset cancer classifications includes: obtaining S input tokens based on the R pieces of adjusted sub-object feature information of the object data under the R feature extraction units respectively, S being a positive integer greater than 1; determining, for an s^{th} input token among the S input tokens by using the gating unit, a probability value indicating that the s^{th} input token is assigned to each of the E expert units, E being a positive integer greater than 1, and s taking values of 1, 2, . . . , S; selecting, from the E expert units based on the probability value, T expert units configured to process the s^{th} input token, and respectively processing the s^{th} input token by using the T expert units, to obtain fused feature information of the s^{th} input token, T being a positive integer less than E; obtaining mixed feature information of the object data based on fused feature information of the S input tokens; and as shown in FIG. 17, performing classification based on the mixed feature information to obtain the probability reference values indicating that the classification object belongs to the plurality of cancer classifications.

For a implementation process of S505 above, refer to the related description of S205 above. Details are not described herein again.

In some embodiments of this application, interrelationships among different gene mutations are extracted by using the MoE module, and interpretability is taken into consideration, which helps to discover potential unknown mutations of cancer types. In some embodiments of this application, starting from two dimensions, namely a patient dimension and a gene mutation state, a deep association relationship within different patients and different gene mutation states is learned, to achieve classification of different types of cancers and obtain classification reference results indicating that the classification object belongs to a plurality of different types of cancers, thereby improving a detection range and detection accuracy of early-stage cancers.

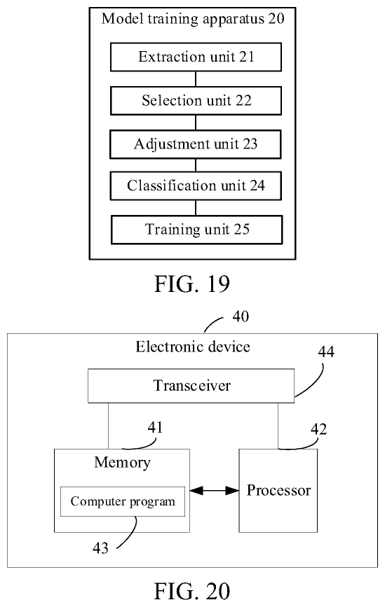

The method embodiments of this application are described in detail above with reference to FIG. 2 to FIG. 17, and apparatus embodiments of this application are described in detail below with reference to FIG. 18 and FIG. 19.

FIG. 18 is a schematic block diagram of a data classification apparatus according to some embodiments.

As shown in FIG. 18, the data classification apparatus 10 includes:
an extraction unit 11, configured to: acquire object data and Q pieces of candidate data, and respectively extract object feature information of the object data and candidate feature information of the Q pieces of candidate data, the object data including parameter values of L biological state parameters of a classification object, the candidate data including parameter values of L biological state parameters of a reference object different from the classification object, and both Q and L being positive integers greater than 1;
a selection unit 12, configured to select, from the Q pieces of candidate data based on the object feature information and the candidate feature information, at least one piece of target candidate data satisfying a preset correlation condition with the object data;
an adjustment unit 13, configured to adjust the object feature information based on the candidate feature information of the at least one piece of target candidate data to obtain adjusted object feature information of the object data; and
a classification unit 14, configured to classify the adjusted object feature information to obtain a classification result of the object data, the classification result including probability reference values indicating that the classification object belongs to a plurality of preset classifications.

In some embodiments, the L biological state parameters include L gene mutations, and the classification result includes probability reference values indicating that the classification object belongs to a plurality of cancer classifications.

In some embodiments, the classification unit 14 classifies the adjusted object feature information of the object data by using a data classification model, the data classification model including a retrieval module and a classification module, the retrieval module including R feature extraction units, R being a positive integer greater than 1, the object feature information includes R pieces of sub-object feature information, and the candidate feature information includes R pieces of sub-candidate feature information; the extraction unit 11 is configured to: perform feature extraction on the object data by using a j^{th} feature extraction unit, to obtain a j^{th} piece of sub-object feature information of the object data, and respectively perform feature extraction on the Q pieces of candidate data by using the j^{th} feature extraction unit, to obtain a j^{th} piece of sub-candidate feature information of the Q pieces of candidate data, j taking values 1, 2, . . . , R; the selection unit 12 is configured to select, from the Q pieces of candidate data based on the R pieces of sub-object feature information of the object data and the R pieces of sub-candidate feature information of the Q pieces of candidate data, Kj pieces of target candidate data satisfying the preset correlation condition with the object data, Kj being a positive integer; the adjustment unit 13 is configured to adjust the j^{th} piece of sub-object feature information of the object data based on a j^{th} piece of sub-candidate feature information of the Kj pieces of target candidate data to obtain a j^{th} piece of adjusted sub-object feature information of the object data; and the classification unit 14 is configured to classify, by using the classification module, R pieces of adjusted sub-object feature information of the object data to obtain the classification result of the object data.

In some embodiments, the R feature extraction units are R encoders, and the extraction unit 11 is configured to: perform feature extraction on the object data by using a j^{th} encoder, to obtain the j^{th} piece of sub-object feature information of the object data, and respectively perform feature extraction on the Q pieces of candidate data by using the j^{th} encoder, to obtain the j^{th} piece of sub-candidate feature information of the Q pieces of candidate data.

In some embodiments, the selection unit 12 is configured to: determine a similarity between the object data and each of the Q pieces of candidate data based on the R pieces of sub-object feature information of the object data and the R pieces of sub-candidate feature information of the Q pieces of candidate data; and select the Kj pieces of target candidate data from the Q pieces of candidate data based on the similarity. The similarity is configured for representing a correlation between the object data and the candidate data.

In some embodiments, the selection unit 12 is configured to: project, for a q^{th} piece of candidate data among the Q pieces of candidate data, a j^{th} piece of sub-candidate feature information of the q^{th} piece of candidate data by using a first projection matrix, to obtain a j^{th} piece of projection feature information of the q^{th} piece of candidate data, q taking values 1, 2, . . . , Q; project the j^{th} piece of sub-object feature information of the object data by using the first projection matrix, to obtain the j^{th} piece of projection feature information of the object data; and determine a distance between R pieces of projection feature information of the object data and R pieces of projection feature information of the q^{th} piece of candidate data, and determine a similarity between the object data and the q^{th} piece of candidate data based on the distance.

In some embodiments, the adjustment unit 13 is configured to: determine, for a k^{th} piece of target candidate data among the Kj pieces of target candidate data, a distance between the j^{th} piece of projection feature information of the object data and a j^{th} piece of projection feature information of the k^{th} piece of target candidate data, and determine first adjustment information of the k^{th} piece of target candidate data for the object data based on the distance, k taking values 1, 2, . . . , Kj; and adjust the j^{th} piece of sub-object feature information of the object data based on respective first adjustment information of the Kj pieces of target candidate data for the object data, to obtain the j^{th} piece of adjusted sub-object feature information of the object data.

In some embodiments, the adjustment unit 13 is configured to: perform linear activation processing on the distance, then perform dropout processing, and perform lossless linear transformation on a result of the dropout processing, to obtain second adjustment information of the k^{th} piece of target candidate data for the object data; and determine the first adjustment information of the k^{th} piece of target candidate data for the object data based on the second adjustment information of the k^{th} piece of target candidate data for the object data and label information of the k^{th} piece of target candidate data.

In some embodiments, the adjustment unit 13 is configured to: project the label information of the k^{th} piece of target candidate data by using a second projection matrix, to obtain label projection information of the k^{th} piece of target candidate data; and determine a sum of the second adjustment information of the k^{th} piece of target candidate data for the object data and the label projection information of the k^{th} piece of target candidate data as the first adjustment information of the k^{th} piece of target candidate data for the object data.

In some embodiments, the adjustment unit 13 is configured to: determine, for a k^{th} piece of target candidate data among the Kj pieces of target candidate data, a weight of the k^{th} piece of target candidate data based on a similarity between the object data and the k^{th} piece of target candidate data; perform, based on weights of the Kj pieces of target candidate data, weighted processing on the first adjustment information of the Kj pieces of target candidate data for the object data, to obtain weighted adjustment information; and determine, based on the j^{th} piece of sub-object feature information of the object data and the weighted adjustment information, the j^{th} piece of adjusted sub-object feature information of the object data.

In some embodiments, the classification module includes a MoE module, the MoE module includes a gating unit and E expert units, and the classification unit 14 is configured to: obtain S input tokens based on the R pieces of adjusted sub-object feature information of the object data, S being a positive integer greater than 1; determine, for an s^{th} input token among the S input tokens by using the gating unit, a probability value indicating that the s^{th} input token is assigned to each of the E expert units, E being a positive integer greater than 1, and s taking values 1, 2, . . . , S; select, from the E expert units based on the probability value, T expert units configured to process the s^{th} input token, and respectively process the s^{th} input token by using the T expert units, to obtain fused feature information of the s^{th} input token, T being a positive integer less than E; obtain mixed feature information of the object data based on fused feature information of the S input tokens; and classify the object data based on the mixed feature information to obtain the classification result of the object data.

In some embodiments, the classification unit 14 is configured to: for each of the T expert units, perform, by using the expert unit, feature fusion processing on input tokens assigned to the expert unit, to obtain output feature information of the input tokens, the input tokens including the s^{th} input token; determine, based on the probability value indicating that the s^{th} input token is assigned to each of the E expert units, weights respectively corresponding to the T expert units; and perform, based on the weights respectively corresponding to the T expert units, weighted processing on output feature information of the s^{th} input token respectively outputted by the T expert units, to obtain the fused feature information of the s^{th} input token.

In some embodiments, the classification unit 14 is configured to: take each of the R pieces of adjusted sub-object feature information of the object data as a row of a first matrix, to obtain the first matrix including R rows; and take each column of the first matrix as one input token, to obtain the S input tokens.

The apparatus embodiments and the method embodiments may correspond to each other. For similar descriptions, refer to the method embodiments. The apparatus shown in FIG. 18 may perform the foregoing embodiments of the data classification method, and the foregoing and other operations and/or functions of the modules in the apparatus are respectively for implementing the foregoing method embodiments.

FIG. 19 is a schematic block diagram of a model training apparatus according to some embodiments.

As shown in FIG. 19, the model training apparatus 20 may include:
an extraction unit 21, configured to: acquire a training sample and Q candidate samples, and respectively extract object feature information of the training sample and candidate feature information of the Q candidate samples by using a retrieval module in a data classification model, the training sample including parameter values of L biological state parameters of a first object, the candidate samples including parameter values of L biological state parameters of a reference object different from the first object, and both Q and L being positive integers greater than 1;
a selection unit 22, configured to select, from the Q candidate samples based on the object feature information and the candidate feature information, at least one target candidate sample satisfying a preset correlation condition with the training sample;
an adjustment unit 23, configured to adjust the object feature information based on the candidate feature information of the at least one target candidate sample to obtain adjusted object feature information of the training sample;
a classification unit 24, configured to classify, by using a classification module in the data classification model, the adjusted object feature information of the training sample to obtain a classification result of the training sample, the classification result including probability reference values indicating that the first object belongs to a plurality of preset classifications; and
a training unit 25, configured to: determine a loss of the data classification model based on the classification result of the training sample, and train the data classification model based on the loss.

In some embodiments, the retrieval module includes R feature extraction units, R being a positive integer greater than 1, the object feature information includes R pieces of sub-object feature information, and the candidate feature information includes R pieces of sub-candidate feature information; the extraction unit 21 is configured to: perform, for a j^{th} feature extraction unit among the R feature extraction units, feature extraction on the training sample by using the j^{th} feature extraction unit, to obtain a j^{th} piece of sub-object feature information of the training sample, and respectively perform feature extraction on the Q candidate samples by using the j^{th} feature extraction unit, to obtain a j^{th} piece of sub-candidate feature information of the Q candidate samples, j taking values 1, 2, . . . , R; the selection unit 22 is configured to select, from the Q candidate samples based on the R pieces of sub-object feature information of the training sample and the R pieces of sub-candidate feature information of the Q candidate samples, Kj target candidate samples satisfying the preset correlation condition with the training sample; the adjustment unit 23 is configured to adjust the j^{th} piece of sub-object feature information of the training sample based on a j^{th} piece of sub-candidate feature information of the Kj target candidate samples to obtain a j^{th} piece of adjusted sub-object feature information of the training sample; and the classification unit 24 is configured to classify, by using the classification module, R pieces of adjusted sub-object feature information of the training sample to obtain the classification result of the training sample.

In some embodiments, the classification module includes a MoE module, the MoE module including a gating unit and E expert units, and the classification unit 24 is configured to: obtain S input tokens based on the R pieces of adjusted sub-object feature information of the object data, S being a positive integer greater than 1; determine, for an s^{th} input token among the S input tokens by using the gating unit, a probability value indicating that the s^{th} input token is assigned to each of the E expert units, E being a positive integer greater than 1, and s being a positive integer less than or equal to S; select, from the E expert units based on the probability value, T expert units configured to process the s^{th} input token, and respectively process the s^{th} input token by using the T expert units, to obtain fused feature information of the s^{th} input token, T being a positive integer less than E; obtain mixed feature information of the object data based on fused feature information of the S input tokens; and classify the object data based on the mixed feature information to obtain the classification result of the object data.

In some embodiments, the training unit 25 is configured to: determine a first loss based on the classification result of the training sample and label information of the training sample; determine a second loss based on a quantity of input labels assigned to each of the E expert units and an assignment probability value; and determine the loss of the data classification model based on the first loss and the second loss.

The apparatus embodiments and the method embodiments may correspond to each other. For similar descriptions, refer to the method embodiments. The apparatus shown in FIG. 19 may perform the foregoing embodiments of the model training method, and the foregoing and other operations and/or functions of the modules in the apparatus are respectively for implementing the foregoing method embodiments.

The apparatus in some embodiments of this application is described above with reference to the accompanying drawings from the perspective of functional modules. The functional modules may be implemented in a form of hardware, or may be implemented in a form of software, or may be implemented in a combination of hardware and software modules. The steps of the method embodiments in the embodiments of this application may be implemented by using an integrated logic circuit of hardware and/or an instruction in a form of software in a processor. The steps of the method disclosed with reference to the embodiments of this application may be directly embodied as being executed by a hardware decoding processor or executed by a combination of hardware and software modules in a decoding processor. In some embodiments, the software module may be located in a mature storage medium in the art, such as a random access memory (RAM), a flash memory, a read-only memory (ROM), a programmable read-only memory (PROM), an electrically-erasable programmable memory, or a register. The storage medium is located in the memory. The processor reads information in the memory and completes the steps in the foregoing method embodiments in combination with hardware thereof.

FIG. 20 is a schematic block diagram of a structure of an electronic device according to some embodiments. The electronic device may be the foregoing terminal device or server.

As shown in FIG. 20, the electronic device 40 may include:
a memory 41 and a processor 42. The memory 41 is configured to store a computer program 43 and transmit the program code 43 to the processor 42. In other words, the processor 42 may invoke and run the computer program 43 from the memory 41, to implement the methods in the embodiments of this application.

For example, the processor 42 may be configured to perform operations in the foregoing methods according to instructions in the computer program 43.

In some embodiments of this application, the processor 42 may include, but is not limited to,
a general-purpose processor, a digital signal processor (DSP), an application integrated circuit (ASIC), a field programmable gate array (FPGA) or another programmable logic device, a discrete gate or transistor logic device, a discrete hardware component, and the like.

In some embodiments of this application, the processor 41 includes, but is not limited to,
a volatile memory and/or a non-volatile memory. The non-volatile memory may be a ROM, a PROM, an erasable PROM (EPROM), an electrically EPROM (EEPROM), or a flash memory. The volatile memory may be a RAM, serving as an external cache. By way of illustration and not limitation, RAMs in many forms may be used, for example, a static RAM (SRAM), a dynamic RAM (DRAM), a synchronous DRAM (SDRAM), a double data rate SDRAM (DDR SDRAM), an enhanced SDRAM (ESDRAM), a synchlink DRAM (SLDRAM), and a direct rambus RAM (DR RAM).

In some embodiments of this application, the computer program 43 may be split into one or more modules. The one or more modules are stored in the memory 41 and executed by the processor 42, to complete the methods provided in this application. The one or more modules may be a series of computer program instruction segments that can implement functions, and the instruction segments are configured for describing an execution process of the computer program 43 in the electronic device.

As shown in FIG. 20, the electronic device 40 may further include:
a transceiver 44. The transceiver 44 may be connected to the processor 42 or the memory 41.

The processor 42 may control the transceiver 44 to communicate with another device and may transmit information or data to another device or receive information or data transmitted by another device. The transceiver 44 may include a transmitter and a receiver. The transceiver 44 may further include an antenna, and there may be one or more antennas.

Components in the electronic device 40 are connected by using a bus system. In addition to a data bus, the bus system further includes a power bus, a control bus, and a state signal bus.

According to an aspect of this application, a computer storage medium is provided. The computer storage medium stores a computer program. The computer program, when executed by a computer, enables the computer to perform the methods in the foregoing method embodiments. Some embodiments further provides a computer program product including instructions. The instructions, when executed by a computer, cause the computer to perform the methods in the foregoing method embodiments.

According to another aspect of this application, a computer program product or a computer program is provided. The computer program product or the computer program includes computer instructions. The computer instructions are stored in a computer-readable storage medium. A processor of a computer device reads the computer instructions from the computer-readable storage medium. The processor executes the computer instructions, to cause the computer device to perform the methods in the foregoing method embodiments.

In other words, when the software is used for implementation, implementation may be entirely or partially performed in a form of a computer program product. The computer program product includes one or more computer instructions. When the computer program instructions are loaded and executed on the computer, the procedures or functions according to the embodiments of this application are all or partially generated. The computer may be a general-purpose computer, a special-purpose computer, a computer network, or another programmable device. The computer instructions may be stored in a computer-readable storage medium, or transmitted from a computer-readable storage medium to another computer-readable storage medium. For example, the computer instructions may be transmitted from a website, computer, server, or data center to another website, computer, server, or data center in a wired (for example, a coaxial cable, an optical fiber, or a digital subscriber line (DSL)) or wireless (for example, infrared, radio, or microwave) manner. The computer-readable storage medium may be any usable medium accessible to the computer, or may be a data storage device such as a server or a data center in which one or more usable media are integrated. The usable medium may be a magnetic medium (for example, a floppy disk, a hard disk, or a magnetic tape), an optical medium (for example, a digital versatile disc (DVD)), a semiconductor medium (for example, a solid state disk (SSD)), or the like.

A person of ordinary skill in the art can be aware that the exemplary modules and algorithm operations described with reference to the embodiments disclosed in this specification can be implemented by electronic hardware or a combination of computer software and electronic hardware. Whether the functions are executed in a mode of hardware or software depends on applications and design constraint conditions of the technical solutions. Those skilled in the art may use different methods to implement the described functions for each application, but such implementation is not to be considered beyond the scope of this application.

In the several embodiments provided in this application, the disclosed system, apparatus, and method may be implemented in other manners. For example, the apparatus embodiments described above are merely exemplary. For example, the module division is merely logical function division and there may be other division during actual implementation. For example, a plurality of modules or components may be combined or integrated into another system, or some features may be ignored or not performed. In addition, the mutual couplings or direct couplings or communication connections that are displayed or discussed may be implemented by using some interfaces. The indirect couplings or communication connections between the apparatuses or modules may be implemented in electronic, mechanical, or other forms.

The modules described as separate components may or may not be physically separated, and the components displayed as modules may or may not be physical modules, and may be located in one place or may be distributed over a plurality of network units. Some or all of the modules may be selected according to actual may achieve the objectives of the solutions of the embodiments. For example, various functional modules in various embodiments of this application may be integrated into one processing module, or various modules may exist physically independently, or two or more modules may be integrated into one module.

The foregoing descriptions are merely implementations of this application, but are not intended to limit the protection scope of this application. Any variation or replacement readily figured out by a person skilled in the art within the technical scope disclosed in this application shall fall within the protection scope of this application. Therefore, the protection scope of this application is subject to the protection scope of the appended claims.

What is claimed is:

1. A data classification method, performed by an electronic device, the method comprising:
acquiring object data of a classification object and Q pieces of candidate data of Q reference objects different from the classification object, wherein the object data includes parameter values of L biological state parameters of the classification object, each of the Q pieces of candidate data includes parameter values of the L biological state parameters of a corresponding reference object of the Q reference objects, and Q and L are each an integer greater than one;
extracting object feature information from the object data and candidate feature information from the Q pieces of candidate data;
selecting, based on the object feature information and the candidate feature information, at least one piece of target candidate data from among the Q pieces of candidate data, the at least one piece of target candidate data satisfying a preset correlation condition with the object data;
adjusting the object feature information using the candidate feature information of the at least one piece of target candidate data to obtain adjusted object feature information of the object data;
classifying the adjusted object feature information to obtain a classification result of the object data, the classification result including probability reference values indicating that the classification object belongs to a plurality of preset classifications; and
displaying, at a terminal device, the classification result as a diagnostic reference for one or more early-stage cancers of the classification object.

2. The method according to claim 1, wherein the L biological state parameters include L gene mutations, and the plurality of preset classifications include a plurality of cancer classifications.

3. The method according to claim 1, wherein the adjusted object feature information is classified using a data classification model, the data classification model including a retrieval module and a classification module, the retrieval module including R feature extraction units, where R is an integer greater than one, the object feature information includes R pieces of sub-object feature information, and the candidate feature information includes R pieces of sub-candidate feature information; and
wherein the extracting includes: performing feature extraction on the object data using a j^{th} feature extraction unit of the R feature extraction units to obtain a j^{th} piece of sub-object feature information of the object data, and performing feature extraction on the Q pieces of candidate data using the j^{th} feature extraction unit to obtain a j^{th} piece of sub-candidate feature information of the Q pieces of candidate data, where j is an integer from one to R;
wherein the selecting includes: selecting, based on the R pieces of sub-object feature information of the object data and the R pieces of sub-candidate feature information of the Q pieces of candidate data, Kj pieces of target candidate data from among the Q pieces of candidate data, the Kj pieces of target candidate data satisfying the preset correlation condition with the object data, where Kj is a positive integer;
wherein the adjusting includes: adjusting the j^{th} piece of sub-object feature information of the object data using a j^{th} piece of sub-candidate feature information of the Kj pieces of target candidate data to obtain a j^{th} piece of adjusted sub-object feature information of the object data; and
wherein the classifying includes: classifying, using the classification module, R pieces of adjusted sub-object feature information of the object data to obtain the classification result of the object data.

4. The method according to claim 3, wherein the R feature extraction units are R encoders, and wherein the performing feature extraction includes:
performing feature extraction on the object data using a j^{th} encoder of the R encoders to obtain the j^{th} piece of sub-object feature information of the object data, and performing feature extraction on the Q pieces of candidate data using the j^{th} encoder to obtain the j^{th} piece of sub-candidate feature information of the Q pieces of candidate data.

5. The method according to claim 3, wherein the selecting includes:
determining a similarity between the object data and each of the Q pieces of candidate data based on the R pieces of sub-object feature information of the object data and the R pieces of sub-candidate feature information of the Q pieces of candidate data, wherein the similarity represents a correlation between the object data and the candidate data; and
selecting the Kj pieces of target candidate data from among the Q pieces of candidate data based on the similarity.

6. The method according to claim 5, wherein the determining the similarity includes:
projecting, for a q^{th} piece of candidate data of the Q pieces of candidate data, a j^{th} piece of sub-candidate feature information of the q^{th} piece of candidate data using a first projection matrix to obtain a j^{th} piece of projection feature information of the q^{th} piece of candidate data, where q is an integer from one to Q;
projecting the j^{th} piece of sub-object feature information of the object data using the first projection matrix to obtain the j^{th} piece of projection feature information of the object data; and
determining a distance between the R pieces of projection feature information of the object data and the R pieces of projection feature information of the q^{th} piece of candidate data, and determining the similarity between the object data and the q^{th} piece of candidate data based on the distance.

7. The method according to claim 6, wherein the adjusting the j^{th} piece of sub-object feature information includes:
determining, for a k^{th} piece of target candidate data of the Kj pieces of target candidate data, a distance between the j^{th} piece of projection feature information of the object data and a j^{th} piece of projection feature information of the k^{th} piece of target candidate data, and determining first adjustment information of the k^{th} piece of target candidate data for the object data based on the distance, where k is an integer from one to Kj; and
adjusting the j^{th} piece of sub-object feature information of the object data based on the respective first adjustment information of the Kj pieces of target candidate data for the object data to obtain the j^{th} piece of adjusted sub-object feature information of the object data.

8. The method according to claim 7, wherein the determining the first adjustment information includes:
performing linear activation processing on the distance, performing dropout processing on a result of the linear activation processing, and performing lossless linear transformation on a result of the dropout processing to obtain second adjustment information of the k^{th} piece of target candidate data for the object data; and
determining the first adjustment information of the k^{th} piece of target candidate data for the object data based on the second adjustment information and the label information of the k^{th} piece of target candidate data.

9. The method according to claim 8, wherein the determining the first adjustment information based on the second adjustment information and the label information includes:
projecting the label information of the k^{th} piece of target candidate data using a second projection matrix to obtain label projection information of the k^{th} piece of target candidate data; and
determining a sum of the second adjustment information and the label projection information of the k^{th} piece of target candidate data as the first adjustment information of the k^{th} piece of target candidate data for the object data.

10. The method according to claim 7, wherein the adjusting based on respective first adjustment information includes:
determining, for a k^{th} piece of target candidate data of the Kj pieces of target candidate data, a weight of the k^{th} piece of target candidate data based on a similarity between the object data and the k^{th} piece of target candidate data;
performing weighted processing on the first adjustment information of the Kj pieces of target candidate data for the object data based on respective weights of the Kj pieces of target candidate data to obtain weighted adjustment information; and
determining the j^{th} piece of adjusted sub-object feature information of the object data based on the j^{th} piece of sub-object feature information of the object data and the weighted adjustment information.

11. The method according to claim 3, wherein the classification module includes a Mixture of Experts (MoE) module, the MoE module including a gating unit and E expert units, where E is an integer greater than one, and wherein the classifying includes:
obtaining S input tokens based on the R pieces of adjusted sub-object feature information of the object data, where S is an integer greater than one;
determining, for an s^{th} input token of the S input tokens using the gating unit, a probability value indicating that the s^{th} input token is assigned to each of the E expert units, where s is an integer from one to S;
selecting, based on the probability value, T expert units from among the E expert units configured to process the s^{th} input token, where Tis a positive integer less than E, and processing the s^{th} input token using the T expert units to obtain fused feature information of the s^{th} input token;
obtaining mixed feature information of the object data based on the fused feature information of the S input tokens; and
classifying the mixed feature information to obtain the classification result of the object data.

12. A data classification apparatus, comprising:
at least one memory configured to store program code; and
at least one processor configured to read the program code and operate as instructed by the program code, the program code comprising:
acquiring code configured to cause at least one of the at least one processor to acquire object data of a classification object and Q pieces of candidate data of Q reference objects different from the classification object, wherein the object data includes parameter values of L biological state parameters of the classification object, each of the Q pieces of candidate data includes parameter values of the L biological state parameters of a corresponding reference object of the Q reference objects, and Q and L are each an integer greater than one;
extracting code configured to cause at least one of the at least one processor to extract object feature information from the object data and candidate feature information from the Q pieces of candidate data;
selecting code configured to cause at least one of the at least one processor to select, based on the object feature information and the candidate feature information, at least one piece of target candidate data from among the Q pieces of candidate data, the at least one piece of target candidate data satisfying a preset correlation condition with the object data;
adjusting code configured to cause at least one of the at least one processor to adjust the object feature information using the candidate feature information of the at least one piece of target candidate data to obtain adjusted object feature information of the object data;
classifying code configured to cause at least one of the at least one processor to classify the adjusted object feature information to obtain a classification result of the object data, the classification result including probability reference values indicating that the classification object belongs to a plurality of preset classifications; and
display code configured to cause at least one of the at least one processor to display, at a terminal device, the classification result as a diagnostic reference for one or more early-stage cancers of the classification object.

13. The apparatus according to claim 12, wherein the L biological state parameters include L gene mutations, and the plurality of preset classifications include a plurality of cancer classifications.

14. The apparatus according to claim 12, wherein the adjusted object feature information is classified using a data classification model, the data classification model including a retrieval module and a classification module, the retrieval module including R feature extraction units, where R is an integer greater than one, the object feature information includes R pieces of sub-object feature information, and the candidate feature information includes R pieces of sub-candidate feature information; and
wherein the extracting code is further configured to cause at least one of the at least one processor to: perform feature extraction on the object data using a j^{th} feature extraction unit of the R feature extraction units to obtain a j^{th} piece of sub-object feature information of the object data, and perform feature extraction on the Q pieces of candidate data using the j^{th} feature extraction unit to obtain a j^{th} piece of sub-candidate feature information of the Q pieces of candidate data, where j is an integer from one to R;
wherein the selecting code is further configured to cause at least one of the at least one processor to: select, based on the R pieces of sub-object feature information of the object data and the R pieces of sub-candidate feature information of the Q pieces of candidate data, Kj pieces of target candidate data from among the Q pieces of candidate data, the Kj pieces of target candidate data satisfying the preset correlation condition with the object data, where Kj is a positive integer;
wherein the adjusting code is further configured to cause at least one of the at least one processor to: adjust the j^{th} piece of sub-object feature information of the object data using a j^{th} piece of sub-candidate feature information of the Kj pieces of target candidate data to obtain a j^{th} piece of adjusted sub-object feature information of the object data; and
wherein the classifying code is further configured to cause at least one of the at least one processor to: classify, using the classification module, R pieces of adjusted sub-object feature information of the object data to obtain the classification result of the object data.

15. The apparatus according to claim 14, wherein the R feature extraction units are R encoders, and wherein the extracting code is further configured to cause at least one of the at least one processor to:
perform feature extraction on the object data using a j^{th} encoder of the R encoders to obtain the j^{th} piece of sub-object feature information of the object data, and perform feature extraction on the Q pieces of candidate data using the j^{th} encoder to obtain the j^{th} piece of sub-candidate feature information of the Q pieces of candidate data.

16. The apparatus according to claim 14, wherein the selecting code is further configured to cause at least one of the at least one processor to:
determine a similarity between the object data and each of the Q pieces of candidate data based on the R pieces of sub-object feature information of the object data and the R pieces of sub-candidate feature information of the Q pieces of candidate data, wherein the similarity represents a correlation between the object data and the candidate data; and
select the Kj pieces of target candidate data from among the Q pieces of candidate data based on the similarity.

17. The apparatus according to claim 16, wherein the selecting code is further configured to cause at least one of the at least one processor to:
project, for a q^{th} piece of candidate data of the Q pieces of candidate data, a j^{th} piece of sub-candidate feature information of the q^{th} piece of candidate data using a first projection matrix to obtain a j^{th} piece of projection feature information of the q^{th} piece of candidate data, where q is an integer from one to Q;
project the j^{th} piece of sub-object feature information of the object data using the first projection matrix to obtain the j^{th} piece of projection feature information of the object data; and
determine a distance between the R pieces of projection feature information of the object data and the R pieces of projection feature information of the q^{th} piece of candidate data, and determine the similarity between the object data and the q^{th} piece of candidate data based on the distance.

18. The apparatus according to claim 17, wherein the adjusting code is further configured to cause at least one of the at least one processor to:
determine, for a k^{th} piece of target candidate data of the Kj pieces of target candidate data, a distance between the j^{th} piece of projection feature information of the object data and a j^{th} piece of projection feature information of the k^{th} piece of target candidate data, and determine first adjustment information of the k^{th} piece of target candidate data for the object data based on the distance, where k is an integer from one to Kj; and
adjust the j^{th} piece of sub-object feature information of the object data based on the respective first adjustment information of the Kj pieces of target candidate data for the object data to obtain the j^{th} piece of adjusted sub-object feature information of the object data.

19. The apparatus according to claim 18, wherein the adjusting code is further configured to cause at least one of the at least one processor to:
perform linear activation processing on the distance, perform dropout processing on a result of the linear activation processing, and perform lossless linear transformation on a result of the dropout processing to obtain second adjustment information of the k^{th} piece of target candidate data for the object data; and
determine the first adjustment information of the k^{th} piece of target candidate data for the object data based on the second adjustment information and the label information of the k^{th} piece of target candidate data.

20. A non-transitory computer-readable storage medium, storing computer code which, when executed by at least one processor, causes the at least one processor to at least:
acquire object data of a classification object and Q pieces of candidate data of Q reference objects different from the classification object, wherein the object data includes parameter values of L biological state parameters of the classification object, each of the Q pieces of candidate data includes parameter values of the L biological state parameters of a corresponding reference object of the Q reference objects, and Q and L are each an integer greater than one;
extract object feature information from the object data and candidate feature information from the Q pieces of candidate data;
select, based on the object feature information and the candidate feature information, at least one piece of target candidate data from among the Q pieces of candidate data, the at least one piece of target candidate data satisfying a preset correlation condition with the object data;
adjust the object feature information using the candidate feature information of the at least one piece of target candidate data to obtain adjusted object feature information of the object data;
classify the adjusted object feature information to obtain a classification result of the object data, the classification result including probability reference values indicating that the classification object belongs to a plurality of preset classifications; and
display, at a terminal device, the classification result as a diagnostic reference for one or more early-stage cancers of the classification object.
